SEQUENCE LISTING (4 sequences)

SEQ ID NO: 1 - DNA, 21 bp, synthetic construct
  source 1..21: mol_type = other DNA
aagtcgccgtcttccgccaag

SEQ ID NO: 2 - DNA, 21 bp, synthetic construct
  source 1..21: mol_type = other DNA
gtccagggacccaatcttcga

SEQ ID NO: 3 - DNA, 24 bp, synthetic construct
  source 1..24: mol_type = other DNA
tacgggttgggggacaggaaacat

SEQ ID NO: 4 - DNA, 24 bp, synthetic construct
  source 1..24: mol_type = other DNA
ggggtgtctccaatgcctgcttct

(19) United States
(12) Patent Application Publication
(10) Pub. No.: US 2024/0309319 A1
(43) Pub. Date: Sep. 19, 2024

(54) COMPOSITIONS AND METHODS FOR THE GENERATION OF NEURONS AND USES THEREOF

(71) Applicant: Washington University, St. Louis, MO (US)

(72) Inventors: Andrew Yoo, St. Louis, MO (US); Lucia Capano, St. Louis, MO (US); Seongwon Lee, St. Louis, MO (US)

(21) Appl. No.: 18/576,712

(22) PCT Filed: Jul. 6, 2022
(86) PCT No.: PCT/US22/73466

§ 371 Date: Jan. 4, 2024

Related U.S. Application Data
(60) Provisional application No. 63/218,889, filed on Jul. 6, 2021.

(51) Int. Cl. C12N 5/0793; C12N 15/113; C12N 15/86; G01N 33/50

(52) U.S. Cl. CPC C12N 5/0619; C12N 15/113; C12N 15/86; G01N 33/5058; C12N 2310/141; C12N 2501/01; C12N 2501/13; C12N 2501/385; C12N 2501/60; C12N 2501/65; C12N 2501/999; C12N 2506/1307; C12N 2510/00; C12N 2740/15043; C12N 2830/003

(57) ABSTRACT

Among the various aspects of the present disclosure is the provision of cells that mirror, recapitulate, mimick, or substantially express endogenous tau isoforms and methods of making and using same.

### CROSS REFERENCE TO RELATED APPLICATIONS

This application claims the benefit of U.S. Provisional Application 63/218,889, filed Jul. 6, 2021 the disclosure of which is hereby incorporated by reference in its entirety.

### GOVERNMENT SUPPORT

This invention was made with government support under AG056296 awarded by the National Institutes of Health. The government has certain rights in the invention.

### REFERENCE TO SEQUENCE LISTING

This application contains a Sequence Listing that has been submitted in ASCII format via EFS-Web and is hereby incorporated by reference in its entirety. The ASCII copy, created on Jul. 6, 2022, is named Untitled_ST26.xml, and is 5 kilobytes in size.

### FIELD OF THE TECHNOLOGY

This disclosure generally relates to methods, compositions, kits, and agents useful for generating neurons, conversion of fibroblasts, screening for therapeutics, and treatment of motor neuron diseases.

### BACKGROUND

Tauopathies are adult-onset, neurodegenerative disorders whose shared pathology is intracellular tau protein aggregates. Tau, encoded by the Microtubule Associated Protein Tau (MAPT) gene, produces six protein isoforms whose expression is under strict developmental regulation, with only one isoform (0N3R) present during fetal development but all six present perinatally and maintained throughout lifespan. Tau isoforms are defined by the inclusion or exclusion of three alternatively spliced exons: 2, 3, and 10. Exon 2 and 3 inclusion produces two N-terminal domains (N1 and N2) whereas exon 10 encodes the second imperfect-repeat microtubule-binding domain (R2) among three other repeat domains (R1, R3, R4). The inclusion or exclusion of exon 10 gives rise to 4-repeat (4R) and 3-repeat (3R) tau, respectively, and these two isoform families are expressed at an approximately 1:1 ratio in the healthy adult human brain. Perturbation of the 3R:4R tau ratio contributes to the pathogenesis of various tauopathies, and out of the 53 known familial pathogenic tau mutations, almost 50% are located within or related to the expression of exon 10. Thus, it is critical to establish a human neuron-based system that recapitulates the endogenous tau isoforms seen in the adult brain and tau pathology resulting from 3R:4R dysregulation in patient-derived neurons. The significance of human neuron-based approaches is also highlighted by the fact that humanized mouse tau models lack the 1:1 3R:4R ratio and requires genetic perturbation to achieve 3R:4R tau level comparable to the adult human brain.

The past decade has primarily used neuronal differentiation of induced pluripotent stem cells (iPSCs) as the primary methodology for generating human neurons (iPSC-Ns). While these cells offer a robust means to generate neurons at a large quantity, their utility in studying processes that occur in aged neurons has been limited due to the reversion of the cellular age, and previous studies revealed iPSC-Ns represent fetal stages of development and express only marginal levels of 1N, 2N, and 4R tau expression. To increase 4R tau levels in iPSC-Ns, several experimental perturbations have been used including transgene overexpression, splicing mutations, or extended culturing times (188-365 days). However, these experimental manipulations still fall short of generating neurons with the endogenous ratio of 3R:4R in the adult human brain. Therefore, an alternative strategy.

Thus, there is a need in the art compositions and methods for generating neurons that mirror the endogenous tau expression and optionally harbor familial mutations to increase the ability to study tauopathies.

### SUMMARY

Among the various aspects of the present disclosure is an in vitro disease model for tauopathies as disclosed herein. The in vitro model can help provide new insights into molecular mechanisms that underlie the pathophysiology of various tauopathies, a model for screening therapeutic agents and provide targets for therapeutic intervention in patients.

Accordingly, one aspect of the present disclosure features methods of generating cells that mirror, recapitulate, mimick, or substantially express endogenous tau isoforms (e.g., at a 1:1 3R:4R tau isoform ratio), methods of using, and compositions comprising the same.

An aspect of the present disclosure provides for a method of modeling a neurodegenerative disease or a method of generating a neuron (e.g., a miRNA-induced neuron (miN)) from an adult somatic cell.

In some embodiments, the method generally includes (i) providing an adult somatic cell, at least one miRNA capable of providing access to motor neuron genes in the adult somatic cell, and/or transcription factors; (ii) providing the at least one miRNA to the adult somatic cell; (iii) providing the transcription factors to the adult somatic cell, resulting in a transduced adult somatic cell; and/or (iv) providing a NEUROD1-activator (e.g., ISX9) to the transduced adult somatic cell. In some embodiments, the methods results in the conversion of the adult somatic cell into a converted neuron. In some embodiments, the transcription factors are selected from the group consisting of: motor neuron transcription factors ISL1 and/or LHX3; or striatal-enriched factors CTIP2, DLX1, DLX2, and/or MYT1L (CDM). In some embodiments, the adult somatic cell is an adult human fibroblast of mesodermal origin. In some embodiments, the miRNA is selected from miR-9/9* and/or miR-124 (miR-9/9*-124). In some embodiments, the adult somatic cells are fibroblasts, the fibroblasts are transduced with supernatant lentivirus mix comprised of dox-inducible miR-9/9*-124, rtTA, and/or the transcription factor MYT1L. In some embodiments, from about PID1 to PID14 cells are treated with DAPT (e.g., about 2 uM) to increase neurite outgrowth and/or neuronal differentiation. In some embodiments, from about PID 3, 6, 10, and/or 14, cells are treated with a NEUROD1-activator, optionally ISX9 (e.g., about 10 uM) to push cortical fate. In some embodiments, the cells (fibroblasts and/or reprogramming cells) are cultured in DMEM+˜10% FBS through replating at ˜PID5. In some embodiments, on about PID6, cells are switched to Neurobasal-A with B27+ (e.g., ˜1000×) and/or Glutamax (e.g., ˜500×), containing ˜1 μg/mL doxycycline, ˜200 μM dibutyl cyclic AMP, ˜1 mM valproic acid, ˜2 uM DAPT, ˜200 nM Ascorbic Acid, ˜10 ng/mL BDNF, ˜10 ng/mL NT-3, ˜1 μM retinoic acid, ˜10 uM ISX9, ˜100× RevitaCell Supplement (RVC), and/or ˜3 μg/mL puromycin. In some embodiments, the cells are half-fed every 4 days and/or doxed every 4 days on an offsetting 2 day scheduled. In some embodiments, on about DIV14, miNs are half fed using BrainPhys containing N2A and/or SM1 (StemCell) with the following: ˜1 μg/mL doxycycline, ˜200 μM dibutyl cyclic AMP, ˜1 mM valproic acid, ˜2 uM DAPT, ˜200 nM Ascorbic Acid, ˜10 ng/mL BDNF, ˜10 ng/mL NT-3, and/or ˜1 μM retinoic acid.

In some embodiments, the miRNA-induced neurons (miNs) recapitulate the expression of all six tau isoforms expressed in adult brains, with 4R tau establishing the 1:1 ratio with 3R tau. In some embodiments, the miRNA-induced neurons (miNs) express endogenously 3R and/or 4R-tau levels analogous to an human adult brain. In some embodiments, the miRNA-induced neurons (miNs) mirror the endogenous tau isoforms in health or disease (e.g., taupathy). In some embodiments, the miRNA-induced neurons (miNs) express the 4 repeat (4R) tau isoform. In some embodiments, the miRNA-induced neurons (miNs) miNs express all six isoforms of tau having 3R/4R isoform ratio substantially equivalent to that detected in human adult brains. In some embodiments, the miRNA-induced neurons (miNs) recapitulate tauopathy having increased 4R tau and/or the formation of insoluble tau with seeding activities. In some embodiments, the converted neuron is a motor neuron or a medium spiny neuron (MSN). In some embodiments, the miRNA or the transcription factors are expressed in the adult somatic cell comprising an adult somatic cell genome by viral vector transduction. In some embodiments, a viral vector expresses miRNA and/or an anti-apoptotic gene, beneficial for neuronal conversion, under an inducible promoter. In some embodiments, the miRNA or the transcription factors are cloned into a lentiviral plasmid; a lentivirus comprising a lentivirus genome is produced and/or the adult somatic cell is infected; the lentivirus genome comprises the miRNA or the transcription factors and/or is transfected into the adult somatic cell genome, resulting in a transduced adult somatic cell; and/or the miRNA or the transcription factors are stably expressed by the transduced adult somatic cell. In some embodiments, the miRNA or the transcription factors are administered exogenously to the adult somatic cell.

In some embodiments, the miRNA coordinates epigenetic and/or transcriptional changes resulting in neuronal cell fate conversion; induces a generic neuronal state characterized by loss of fibroblast identity, presence of a pan-neuronal gene expression program, and/or absence of subtype specificity; initiates subunit switching within BAF chromatin remodeling complexes while separately repressing neuronal cell-fate inhibitors REST, Co-REST, and/or SCP1; or alters expression of genes involved in DNA methylation, histone modifications, chromatin remodeling, and/or chromatin compaction. In some embodiments, the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and/or a glutamatergic neuron. In some embodiments, the converted neuron phenotypically resembles an endogenous motor neuron when compared using immunostaining analysis or gene expression profiling; the converted neuron resembles the endogenous motor neuron when compared using electrophysiological tests or co-culture tests; or the converted neuron retains donor age marks and/or positional information from the adult somatic cell. In some embodiments, the neurodegenerative disease, disorder, or condition is selected from one or more of the group consisting of: (i) a tauopathy; (ii) a motor neuron disease; (iii) spinal cord injury (SCI); (iv) progressive supranuclear palsy (PSP); frontotemporal lobar degeneration (FTLD-TAU); corticobasal degeneration; or Alzheimer disease; (v) Amyotrophic Lateral Sclerosis (ALS) or Spinal Muscular Atrophy (SMA); or (vi) Huntington's Disease (HD) or Alzheimer's Disease (AD). Another aspect of the present disclosure provides for a method of screening a candidate drug for effectiveness in treating a tauopathy, neurodegenerative, or motor neuron disease comprising: (i) providing a cellular platform, the cellular platform comprising neurons generated from fibroblasts of a subject with a neurodegenerative or motor neuron disease according to the method of any one of the preceding claims; (ii) providing a candidate drug; (iii) contacting the candidate drug and/or the cellular platform; and/or (iv) assessing efficacy of the candidate drug. In some embodiments, the cellular platform comprises cells obtained from a subject with a tauopathy, a motor neuron disease, Alzheimer's Disease (AD), Amyotrophic Lateral Sclerosis (ALS), Spinal Muscular Atrophy (SMA), Spinal Cord Injury (SCI), Huntington's Disease (HD), progressive supranuclear palsy (PSP), frontotemporal lobar degeneration (FTLD-TAU), or corticobasal degeneration. In some embodiments, the efficacy is evaluated by monitoring the neurons for reversal of electrical impairment, spontaneous cell death, or stress-induced cell death. In some embodiments, the subject has or is suspected of having a motor neuron disease, Alzheimer's Disease (AD), Amyotrophic Lateral Sclerosis (ALS), Spinal Muscular Atrophy (SMA), Spinal Cord Injury (SCI), or Huntington's Disease (HD). In some embodiments, the subject has or is suspected of having a tauopathy. In some embodiments, the subject has or is suspected of having a tauopathy selected from progressive supranuclear palsy (PSP); frontotemporal lobar degeneration (FTLD-TAU); corticobasal degeneration; or Alzheimer disease. In some embodiments, the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and/or a glutamatergic neuron. Another aspect of the present disclosure provides for a reprogrammed human neuron having a ratio (e.g., 1:1, non-1:1) of 4 repeat (4R) and/or 3 repeat (3R) isoforms consistent with healthy or pathological brain tissue generated according to any one of the preceding claims.

In another aspect the present disclosure provides for methods of modeling a neurodegenerative disease or methods of generating a neuron from an adult somatic cell, the methods generally comprise, (i) providing an adult somatic cell, at least one miRNA capable of providing access to motor neuron genes in the adult somatic cell, and transcription factors; (ii) providing the at least one miRNA to the adult somatic cell; (iii) providing the transcription factors to the adult somatic cell, resulting in a transduced adult somatic cell; and (iv) providing a NEUROD1-activator to the transduced adult somatic cell, resulting in the conversion of the adult somatic cell into a converted neuron. In some embodiments, the transcription factors are selected from motor neuron transcription factors, such as ISL LIM Homeobox 1 (ISL1) and/or LIM Homeobox 3 (LHX3); and/or striatal-enriched factors, such as, COUP-TF-Interacting Protein 2 (CTIP2), Distal-Less Homeobox 1 (DLX1), Distal-Less Homeobox 2 (DLX2), and/or Myelin Transcription Factor 1 Like (MYT1L (CDM)). In some embodiments, the adult somatic cell is an adult human fibroblast of mesodermal origin. In each of the above embodiments, the miRNA is selected from miR-9/9* and miR-124 (miR-9/9*-124). In some embodiments, the adult somatic cells are fibroblasts, the fibroblasts are transduced with supernatant lentivirus mix comprised of dox-inducible miR-9/9*-124, reverse tetracycline-controlled transactivator (rtTA), and the transcription factor MYT1L.

In some embodiments, from about day 1 to day 14 the somatic cells are treated with N—[N-(3,5-Difluorophenacetyl-L-alanyl)]-S-phenylglycine tbutyl ester (DAPT) to increase neurite outgrowth and neuronal differentiation, wherein DAPT is in a concentration from about 0.2 μM to about 4 μM. In some embodiments, on about days 3, 6, 10, and 14, the cells are treated with a NEUROD1-activator to push cortical fate. In certain embodiments, the NEUROD1-activator is Isoxazole 9 (ISX9), where ISX9 is used in a concentration of about 1 μM to about 100 μM.

In some embodiments, the cells are cultured in a medium with Dulbecco's Modified Eagle Medium (DMEM) as the basal medium comprising Fetal Bovien Serum (FBS) through replating at day 5. In some embodiments, on about day 6, the cells are contacted with a cell culture medium wherein the basal medium is Neurobasal-A with B27+, Glutamax, dibutyl cyclic AMP, valproic acid, DAPT, Ascorbic Acid, brain-derived neurotrophic factor (BDNF), Neurotrophin 3 (NT-3), retinoic acid, ISX9, RevitaCell Supplement (RVC), an antibiotic and optionally doxycycline when the expression of miR-9/9*-127 are under control of an inducible tet promoter.

In some embodiments, the cells are half-fed every 4 days and doxed every 4 days on an offsetting 2 day scheduled. In some embodiments, on about day 14, cells are half fed using a culture medium with BrainPhys as the basal medium with Neuro 2A (N2A), STEMCELL Modified-1 (SM1), dibutyl cyclic AMP, valproic acid, DAPT, Ascorbic Acid, BDNF, NT-3, retinoic acid and optionally doxycycline when the expression of miR-9/9*-127 are under control of an inducible tet promoter.

In some embodiments, the miRNA-induced neurons (miNs) recapitulate the expression of all six tau isoforms expressed in adult brains, with 4R tau establishing the 1:1 ratio with 3R tau. In some embodiments, the miRNA-induced neurons (miNs) express endogenously 3R and 4R-tau levels analogous to an human adult brain. In some embodiments, the miRNA-induced neurons (miNs) mirror the endogenous tau isoforms in health and disease (e.g., tauopathy). In some embodiments, the miRNA-induced neurons (miNs) express the 4 repeat (4R) tau isoform. In some embodiments, the miRNA-induced neurons (miNs) miNs express all six isoforms of tau having 3R/4R isoform ratio substantially equivalent to that detected in human adult brains. In some embodiments, the miRNA-induced neurons (miNs) recapitulate tauopathy having increased 4R tau and the formation of insoluble tau with seeding activities.

In some embodiments, the converted neuron is a motor neuron or a medium spiny neuron (MSN). In some embodiments, the miRNA or the transcription factors are expressed in the adult somatic cell comprising an adult somatic cell genome by viral vector transduction. In some embodiments, a viral vector expresses miRNA and an anti-apoptotic gene, beneficial for neuronal conversion, under an inducible promoter. In some embodiments, the miRNA or the transcription factors are cloned into a lentiviral plasmid; a lentivirus comprising a lentivirus genome is produced and the adult somatic cell is infected; the lentivirus genome comprises the miRNA or the transcription factors and is transfected into the adult somatic cell genome, resulting in a transduced adult somatic cell; and the miRNA or the transcription factors are stably expressed by the transduced adult somatic cell. In some embodiments, the miRNA or the transcription factors are administered exogenously to the adult somatic cell. In some embodiments, the miRNA coordinates epigenetic and transcriptional changes resulting in neuronal cell fate conversion; induces a generic neuronal state characterized by loss of fibroblast identity, presence of a pan-neuronal gene expression program, and absence of subtype specificity; initiates subunit switching within BAF chromatin remodeling complexes while separately repressing neuronal cell-fate inhibitors REST, Co-REST, and SCP1; or alters expression of genes involved in DNA methylation, histone modifications, chromatin remodeling, and chromatin compaction.

In some embodiments, the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron. In some embodiments, the converted neuron phenotypically resembles an endogenous motor neuron when compared using immunostaining analysis or gene expression profiling; the converted neuron resembles the endogenous motor neuron when compared using electrophysiological tests or co-culture tests; or the converted neuron retains donor age marks and positional information from the adult somatic cell.

In some embodiments, the neurodegenerative disease, disorder, or condition is selected from (i) a tauopathy; (ii) a motor neuron disease; (iii) spinal cord injury (SCI); (iv) progressive supranuclear palsy (PSP); frontotemporal lobar degeneration (FTLD-TAU); corticobasal degeneration; or Alzheimer disease; (v) Amyotrophic Lateral Sclerosis (ALS) or Spinal Muscular Atrophy (SMA); or (vi) Huntington's Disease (HD) or Alzheimer's Disease (AD).

In another aspect the present disclosure provides methods of screening a candidate drug for effectiveness in treating a tauopathy, neurodegenerative, or motor neuron disease, the methods generally comprise (i) providing a cellular platform, the cellular platform comprising neurons generated from fibroblasts of a subject with a neurodegenerative or motor neuron disease according to the method of the present disclosure; (ii) providing a candidate drug; (iii) contacting the candidate drug and the cellular platform; and (iv) assessing efficacy of the candidate drug. In some embodiments, the cellular platform comprises cells obtained from a subject with a tauopathy, a motor neuron disease, Alzheimer's Disease (AD), Amyotrophic Lateral Sclerosis (ALS), Spinal Muscular Atrophy (SMA), Spinal Cord Injury (SCI), Huntington's Disease (HD), progressive supranuclear palsy (PSP), frontotemporal lobar degeneration (FTLD-TAU), or corticobasal degeneration. In some embodiments, the efficacy is evaluated by monitoring the neurons for reversal of electrical impairment, spontaneous cell death, or stress-induced cell death. In some embodiments, the subject has or is suspected of having a motor neuron disease, Alzheimer's Disease (AD), Amyotrophic Lateral Sclerosis (ALS), Spinal Muscular Atrophy (SMA), Spinal Cord Injury (SCI), or Huntington's Disease (HD). In some embodiments, the subject has or is suspected of having a tauopathy. In some embodiments, the subject has or is suspected of having a tauopathy selected from progressive supranuclear palsy (PSP); frontotemporal lobar degeneration (FTLD-TAU); corticobasal degeneration; or Alzheimer disease.

In some embodiments, the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron.

In still another aspect, the present disclosure provides reprogrammed human neuron having a ratio (e.g., 1:1, non-1:1) of 4 repeat (4R) and 3 repeat (3R) isoforms consistent with healthy or pathological brain tissue generated according to the methods of the present disclosure.

In still yet another aspect, the present disclosure provides methods of generating a neuron cell from a non-neuronal somatic cell, the methods generally comprise i) providing at least one non-neuronal somatic cell; ii) expressing in the non-neuronal somatic cell a MYT1L transcription factor; iii) culturing the cell from step ii) in the presence of a basal medium, serum and DAPT and adding ISX9 to the medium after about 3 days; iv) culturing the cells from step iii) after about 5 days in the presence of a basal medium, B27, glutamax, dibutyl cyclic AMP, valproic acid, DAPT, Ascorbic Acid, BDNF, NT-3, retinoic acid, ISX9, RVC, and expressing in the cells miR-9/9*-127; and v) culturing the cells from step iv) after about 9 days in the in the presence of a basal medium, N2A, SM1, dibutyl cyclic AMP, valproic acid, DAPT, Ascorbic Acid, BDNF, NT-3, and retinoic acid. In some embodiments, the basal medium in step iii) is DMEM and the serum is FBS. In some embodiments, the basal medium in step iv) is Neurobasal-A. In some embodiments, the somatic cells are obtained from a healthy subject. In some embodiments, the somatic cells are obtained from a subject diagnosed with a disease, disorder or at risk of a disease or disorder. In some embodiments, the disease is a CNS disease. In some embodiments, the disease is a tauopathy. In some embodiments, the somatic cell or generated neuron is genetically modified. In some embodiments, the basal medium in step v) is BrainPhys. In some embodiments, the present disclosure provides a cell culture medium comprising the components as described above. Thus, the present disclosure also provides kits for preparing the cell culture medium according to the disclosure having individually packaged components, basal medium, and instructions for preparing the cell culture medium.

In another aspect, the present disclosure provides a population of neurons, which is produced by a method according to the present disclosure. The present disclosure also provides an in vitro cell culture system having (i) a cell culture vessel; and (ii) a population of neurons of the disclosure.

In still another aspect, the present disclosure provides for methods for identifying an agent for treating a tauopathy or CNS disease, the method generally comprise (i) providing an in vitro cell culture system of the disclosure, (ii) culturing the neuron cells in the presence of a candidate agent; and (iii) identifying the candidate agent as an agent for treating a tauopathy or CNS disease, if the candidate agent maintains a cell phenotype or changes a cell phenotype to resemble a healthy control neuron.

Other objects and/or features will be in part apparent and in part pointed out hereinafter.

### BRIEF DESCRIPTION OF THE FIGURES

The patent or patent application file contains at least one drawing executed in color. Copies of this patent or patent application publication with color drawing(s) will be provided by the Office upon request and payment of the necessary fee.

FIG. 1A-1F Directly-reprogrammed neurons display neuronal morphology and transcriptome. FIG. 1A shows representative images of miNs from healthy adult individuals (Healthy 3+4) at post-induction day (PID) 30 immunostained with NCAM (composite of 3×2 40× images stitched together), MAP2, NEFH, and TUBB3. Insets at the corners are immunostaining images performed in starting fibroblasts. Blue rectangular outline depicts the region shown in magnified images. Scale bars=50 μm. FIG. 1B shows representative image of Healthy 4 immunostained for total tau and TUBB3. Inset is starting fibroblasts. Blue rectangular outline depicts the magnified region showing tau islands. Scale bars=50 μm. FIG. 1C shows representative images of healthy miNs stained for tau and TUBB3. Right, quantification of % positive over DAPI in reprogrammed cells from four independent fibroblast samples (MAP2 Number of cells counted: miN 1=140, miN 2=82, miN 3=112, miN 4=169; MAPT Ns: miN 1=113, miN 2=118, miN 3=78, miN 4=87). Scale bars=50 □m. FIG. 1D shows heatmap plots Z-scores comparing qPCR of neuronal genes between miNs and starting fibroblasts from four independent samples. FIG. 1E shows fold change of cortical genes between starting fibroblasts and day 30 miNs. n=3 replicates from miNs grown in three independent wells. Mean+SEM.; two-tailed Student's t-test; *p≤0.05, **p≤0.01, ***p≤0.001. FIG. 1F shows UMAP projection of cells colored by starting cell type (Fibroblast: n=2533 cells; iPSC-N: n=7212 cells; miN: n=9305 cells). Right, UMAP projection of cells colored by non-neuronal (SERPINE1) and pan-neuronal markers (MAPT, MAP2, NCAM1, KIF1A).

FIG. 2A-2G show MiN express exon 10 inclusion at equivalent ratio to the adult human brain. FIG. 2A shows UMAP projection of cells colored by age-associated markers (CDKN1A, CDKN2A, BAG3, and CAV1) and fetal-associated markers (EPHA3 and BCL2). Cell numbers as indicated in FIG. 1F. FIG. 2B shows representative RNA-seq tracks at the MAPT locus of fibroblasts, primary human neurons, cortical iPSC-Ns, adult human brain, and miNs at PID21. FIG. 2C shows Leafcutter analysis of MAPT exon 10 inclusion between fetal neurons and adult brain, and healthy iPSC-Ns and healthy miNs. Percent exon 10 inclusion is quantified on the right. For primary fetal neurons and adult brain, n=sequencing replicates performed from purchased purified RNA (see STAR Methods). For iPSC-Ns, n=replicates grown in three independent wells. For miNs, n=three independent well replicates from three independent individuals. FIG. 2D shows semi-quantitative PCR of 3R and 4R tau isoforms. Right: Quantification of 3R and 4R percent ratio of samples grouped by miNs, fetal samples, and adult brain from independent individuals as indicated in the graph legend. Mean±SEM; One-way ANOVA with multiple comparisons with post hoc Tukey's test; ***p≤0.001. FIG. 2E shows semi-quantitative PCR of 3R and 4R tau expression. Red rectangular outline depicts the time point when 4R tau starts being expressed consistently in multiple samples (n=three independent individuals). FIG. 2F shows MAPT exon inclusion. FIG. 2G shows % N isoforms.

FIG. 3A-3E show MiN tau protein profile mirrors that of the adult human brain. FIG. 3A Quantitation of relative tau peptides from multiple independent samples as indicated in the plot legend. Mean(thick line)±SEM. Pink and red dashed outline depicts 1N, 1N/2N, and 4R-specific tau peptides, respectively. FIG. 3B shows quantification of 3R:4R isoform ratio of all samples used in FIG. 3A. Histogram colors are matched to sample colors shown in the plot legend of FIG. 3A. Mean±SEM; One-Way ANOVA with multiple comparisons and post hoc Tukey's test: Between any fetal vs adult sample for all three 4R peptides: ***p≤0.001. FIG. 3C shows percent of N isoforms (calculation in methods) using all samples as indicated in the plot legend in FIG. 3A. Mean+SEM; One-Way ANOVA with multiple comparisons and post hoc Tukey's test. All N isoforms between fetal vs healthy iPSC-Ns and between adult vs healthy miNs: ns p>0.999; between healthy iPSC-Ns vs healthy miNs: 0N: **p=0.002, 1N: **p=0.002, 2N: *p=0.015. FIG. 3D shows western blot of tau isoforms. FIG. 3E shows western blot of tau isoforms over reprogramming time course. Right, quantification of 3R:4R ratio of 0N and 1N isoforms during the time course of reprogramming of two independent samples indicated in the plot legend. Mean±SEM; One-Way ANOVA with multiple comparisons and post hoc Tukey's test; Between fetal vs any miN: **p=0.002; between fetal and adult: **p=0.004.

FIG. 4A-4F show IVS10+16 patient-derived miNs show increased 4R tau mRNA. FIG. 4A show representative images of reprogrammed IVS10+16 miNs from four independent, symptomatic IVS10+16 patients, immunostained for tau, MAP2 and TUBB3. Right, quantification of % positive over DAPI (MAP2 Number of cells counteds: IVS10+16 1=118, IVS10+16 2=173, IVS10+16 3=105, IVS10+16 4=152; MAPT Ns: IVS10+16 miN 1=98, IVS10+16 miN 2=125, IVS10+16 miN 3=89, IVS10+16 miN 4=88). Scale bars=50 μm. FIG. 4B shows Z score heatmap (from qPCR) representation of neuronal marker gene expression MAP2, NEFL, MAPT, and VGLUT1 in IVS10+16 miNs and starting fibroblasts from four independent lines. FIG. 4C shows LONGO long gene analysis between starting fibroblasts (red) and reprogrammed healthy and IVS10+16 miNs (blue and orange, respectively). FIG. 4D shows Leafcutter analysis of MAPT exon 10 inclusion between healthy and IVS10+16 miNs. Percent exon 10 inclusion is quantified on the right. For miN groups, n=three independent well replicates from three independent individuals. FIG. 4E shows sqPCR for detecting 4R and 3R tau ratio. Right: sqPCR intensity values grouped by healthy miNs, IVS10+16 miNs, and healthy adult brain for 3R and 4R. Mean±SEM. One-Way ANOVA with multiple comparisons and post hoc Tukey's test; Between healthy miNs vs IVS10+16 miNs and IVS10+16 miNs vs adult brain: ***p≤0.001. FIG. 4F shows sqPCR of both healthy and IVS10+16 human brain and miNs.

FIG. 5A-5D show IVS10+16 patient-derived miNs demonstrate increased 4R protein corresponding to increased 4R mRNA. FIG. 5A shows quantitation of relative tau peptides from multiple independent samples as indicated in the plot legend. Mean(thick line)±SEM. FIG. 5B shows quantification of 3R:4R isoform ratio of all samples used in FIG. 5A. Histogram colors are matched to sample colors shown in the plot legend of FIG. 5A. Mean±SEM. One-Way ANOVA with multiple comparisons and post hoc Tukey's test; healthy miNs vs IVS10+16 miNs: 275-280 **p=0.004, 282-290 **p=0.004, 299-317 **p=0.008. Between any miN and iPSC-N: all peptides ***p s 0.001. FIG. 5C shows fold change of 4R isoform expression for all three 4R-specific peptides over healthy miNs. FIG. 5D shows ercentage of N isoforms using all samples as indicated in the plot legend in FIG. 5A. Mean±SEM; One-Way ANOVA with multiple comparisons and post hoc Tukey's test. For all N isoforms: between healthy iPSC-Ns and IVS10+16 iPSC-Ns: ns p>0.999; between healthy miNs vs IVS10+16 miNs: 0N: ns p=0.97; 1N: ns p=0.983; 2N: ns p=0.887; between IVS10+15 miNs vs IVS10+16 iPSC-Ns: 0N: *p=0.038; 1N: *p=0.027; 2N: ns p=0.285.

FIG. 6A-6E show formation of seed-capable and insoluble tau in IVS10+16 patient-derived miNs. FIG. 6A shows representative FRET signals for healthy and IVS10+16 iPSC-N and miN lysate. Right, quantification of % FRET. n=20,000 cells per FRET run. For iPSC-N samples, we performed three independent FRET runs per cell pellet. For miNs, we used three healthy and four IVS10+1 6 independent, individual samples, with three replicate FRET runs per sample. One-way ANOVA with multiple comparisons. *p≤0.05, **p≤0.01. FIG. 6B shows representative images of methanol-fixed healthy and IVS10+16 miNs. Cells were co-labeled with MAP2 antibody (green) and one of five anti-tau antibodies: AT8, CP13, MC1, PHF1, and TOC1 (yellow). Below, quantification of anti-tau antibody signal positive area per 100 nuclei from three healthy and four IVS10+16 independent, individual samples as indicated in plot legend. Nuclei counted per condition: AT8: healthy miN=70939, IVS10+16 miN=84046; CP13: healthy miN=69708, IVS10+16 miN=88775; PHF1: healthy miN=93443, IVS10+16 miN=71070; TOC1: healthy miN=47280, IVS10+16 miN=91376; MC1: healthy miN=94171, IVS10+16 miN=97931. Scale bars=50 μm FIG. 6C shows high magnification of IVS10+16 miNs and insoluble tau threads and tendrils stained with total tau antibody, CP27. Scale bars=50 μm. FIG. 6D shows stimulated emission depletion (STED) microscopy of insoluble tau in IVS10+16 miNs. Scale bars=10 μm. FIG. 6E shows arrowheads indicate the location of insoluble tau signals in healthy miNs (top) and IVS10+16 miNs (bottom). Left images: cells treated with control siRNA (siCtrl). Right images: cell treated with siRNA against total tau (siTotal tau). Right plot: Quantification of tau signals detected per 100 nuclei (DAPI) in healthy miNs (healthy miNs treated with siCtrl) in comparison to IVS10+16 miNs treated with siCtrl and IVS10+16 miNs treated with siTotal tau from three healthy and four IVS10+16 independent, individual samples. Nuclei counted per condition: siCTRL on healthy miNs=140,283; siCTRL on IVS10+16 miNs=117,781; siTau on IVS10+16 miNs=114,050. One-way ANOVA with multiple comparisons. ***p≤0.001. Scale bars=50 μm.

FIG. 7 shows the experimental design and results of Example 1.

### DETAILED DESCRIPTION

Strategies to generate human neurons that mirror the endogenous tau expression and harbor familial mutations have been elusive but will greatly increase the ability to study tauopathies. The present disclosure is based, at least in part, on the discovery that miRNA-mediated direct reprogramming of somatic cells generates human neurons that express both 3R and 4R tau in the same ratio detected in adult human brains. RNA profiling by transcript assays and protein profiling by mass spectrometry demonstrates that adult human brain and miNs from adult fibroblasts have indistinguishable 4R tau profiles, which starkly contrasts with the low 4R tau expression in primary fetal human neurons and iPSC-Ns. The endogenous tau isoform regulation in miNs is sensitive to a point mutation within the splice site proximal to exon 10 in tauopathy patients resulting in reprogrammed neurons that showed increased 4R:3R tau ratio. Significantly, the increased level of 4R tau correlated with the formation of insoluble tau and seed-competent tau, reminiscent of that seen in human 4R tauopathies, and contrasting the lack of seed-competent tau seen in iPSC-Ns harboring the same patient mutation. Thus, the present disclosure provides, in part, a protocol for miRNA-mediated neuronal reprogramming, a robust model for studying both the normal and abnormal biology of tau, exon 10 pathological mutations, a model for screening therapeutic agents and provide targets for therapeutic intervention in patients. As the knowledge of disease-causing mechanisms further accumulates, it would be relied on to predict the clinical course from the genotype and design personalized management strategies at an early stage of the disease.

### I. Method of Producing Neurons from Non-Neuronal Somatic Cells

Methods of converting non-neuronal somatic cells into induced neuronal cells are provided. Aspects of the methods include contacting a non-neuronal somatic cell with a microRNA-mediated neuronal cell induction agent, a NEUROD1-activator and transducing the somatic cell with one or more transcription factors. Aspects of the disclosure further include compositions produced by methods of the disclosure as well as compositions that find use in practicing embodiments of methods of disclosure methods and compositions find use in a variety of different applications.

Tauopathy is a pathogenic process that underlies many forms of neurodegenerative disorders including Alzheimer's disease and primary tauopathy. A key requirement to properly study the adult-onset tauopathy is the ability to replicate the expression of tau isoforms typically seen in adult human brains. Whether one can generate human neurons that express the 4 repeat (4R) tau isoform, in particular, has been a question of paramount importance, since adult human brain forms the 1:1 ratio between 3R and 4R tau isoforms and perturbation of this ratio has been implicated in the onset of tau aggregation and tauopathy. So far, this has remained a challenge as neurons differentiated from induced pluripotent stem cells (iPSCs) express primarily 3R tau as iPSC-derived neurons reflect the fetal age, and there has been no demonstration of reprogrammed human neurons that precisely mirror the 3R and 4R ratio at the level similar to the human adult brain.

This disclosure overcomes the difficulty of generating human neurons that express tau isoforms analogous to the human adult brain. This is a critical feature for studying adult-onset tauopathies.

Aspects described herein stem from, at least in part, development of methods that efficiently direct generation of non-neural somatic cells into neurons. In particular, the present disclosure provides, inter alia, an in vitro culturing process for producing a population of neurons from non-neural somatic cells and the resultant neurons express tau isoforms analogous to the human adult brain, from unmodified healthy somatic cell (e.g., from a healthy human subject). In some embodiments, this culturing process may involve multiple culturing stages (e.g., 2, 3, or more). In some embodiment, the total time period for the in vitro culturing process described herein can range from about 12 days or more. In an exemplary embodiment, the production time period is at least 14 days.

Non-neuronal somatic cells include any somatic cell that would not give rise to a neuron in the absence of experimental manipulation. Non-limiting examples of non-neuronal somatic cells include differentiating or differentiated cells from ectodermal (e.g., keratinocytes), mesodermal (e.g. fibroblast), endodermal (e.g., pancreatic cells), or neural crest lineages (e.g. melanocytes). The somatic cells may be, for example, pancreatic beta cells, glial cells (e.g. oligodendrocytes, astrocytes), hepatocytes, hepatic stem cells, cardiomyocytes, skeletal muscle cells, smooth muscle cells, hematopoietic cells, osteoclasts, osteoblasts, pericytes, vascular endothelial cells, Schwann cells, dermal fibroblasts, and the like. They may be terminally differentiated cells, or they may be capable of giving rise to cells of a specific, non-neuronal lineage, e.g. cardiac stem cells, hepatic stem cells, and the like. The somatic cells are readily identifiable as non-neuronal by the absence of neuronal-specific markers that are well-known in the art, and described herein. Of interest are cells that are vertebrate cells, e.g., mammalian cells, such as human cells, including adult human cells. In certain embodiments, human fibroblasts are preferred. In some instances, the non-neuronal somatic cells are glial cells (glia). The terms “glia” or “glial cells” refer to non-neuronal cells found in close contact with neurons, and encompass a number of different cells, including but not limited to the microglia, macroglia, neuroglia, astrocytes, astroglia, oligodendrocytes, ependymal cells, radial glia, Schwann cells, satellite cells, and enteric glial cells. Examples of markers that may be used to aid in the identification of glial cells include, but are not limited to Glial Fibrillary Acidic Protein (GFAP), 2′,3′-cyclic nucleotide 3′ phosphodiesterase (CNPase), myelin-associated glycoprotein (MAG), myelin basic protein (MBP), and S100 calcium binding protein B (s100B).

The somatic cells may be obtained or previously obtained from any mammal, including humans, primates, domestic and farm animals, and zoo, laboratory or pet animals, such as dogs, cats, cattle, horses, sheep, pigs, goats, rabbits, rats, mice etc. They may be established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

The subject cells may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, adipose, and other differentiated tissues. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Aspects of these methods include contacting a non-neuronal somatic cell (or collection of non-neuronal somatic cells, e.g., culture or a present in a tissue of an organism) with a microRNA useful for induction to a neuronal cell, where the microRNA is provided in an amount sufficient to cause microRNA-mediated conversion of the non-neuronal somatic cell into an induced neuronal cell. The specific nature of the induction agent may vary depending on the particular embodiment of the methods being practiced. Non-limiting examples include nucleic acids (e.g., microRNA or expression cassettes that encode the same, where the expression cassettes may be present in a vector), expression inducers, polypeptides, small molecules, and combinations thereof.

In some instances, the microRNA-mediated conversion that is caused by the induction agent includes providing a level of two or more microRNAs in the cell that is sufficient to cause the cell to convert to an induced neuronal cell. In other words, contact of the cell with the agent results in a level or concentration of two or more microRNAs, such as two distinct microRNAs, which is sufficient (i.e., at a value that) to cause conversion of the cell into a neuronal cell. In some instances, the induction agent is one that causes the level of two or more microRNAs in a cell to be sufficient to cause the cell to convert to an induced neuronal cell.

In one such embodiment, a first microRNA of interest is miR-9/9*. The sequence of miR-9/9* is reported at “mirbase.org.” See also Yoo, A. S., Staahl, B. T., Chen, L, & Crabtree, G. R., MicroRNA-mediated switching of chromatin-remodeling complexes in neural development. Nature 460 (7255), 642-646 (2009). In one such embodiment, a second microRNA of interest is miR-124. The sequence miR-124 is reported at “mirbase.org.” See also Yoo, A. S., Staahl, B. T., Chen, L, & Crabtree, G. R., MicroRNA-mediated switching of chromatin-remodeling complexes in neural development. Nature 460 (7255), 642-646 (2009) Accordingly, in some instances, the agent is one that, upon contact with the non-neuronal somatic cell, causes a level of one or more of miR-9/9* and miR-124 to be present in the cell that is sufficient to cause the cell to convert to a neuronal cell. While the level that is achieved by a given agent may vary, the level may be 25% or more, such as 50% or more, including 75% or more (e.g., 90% or more) of that observed in neurons derived from brain tissue, e.g., as determined via any convenient protocol, such as RT-PCR.

Thus, in certain embodiments, neuronal microRNAs, such as miR-9/9* and miR-124 (miR-9/9*-124) to direct cell-fate conversion of adult human fibroblasts to post-mitotic neurons and, with additional transcription factors, enable the generation of discrete neuronal subtypes. Previously, the molecular events underlying the neurogenic switch mediated by microRNAs during neuronal reprogramming were unknown. Here, the neurogenic state induced by miR-9/9*-124 expression alone was systematically dissected alone and reveal the surprising capability of miR-9/9*-124 in coordinately stimulating the reconfiguration of chromatin accessibility, DNA methylation and mRNA levels, leading to the generation of functionally excitable miRNA-induced neurons, yet uncommitted towards a particular subtype-lineage.

As described herein, it was discovered that micro RNAs (miRNAs) (e.g., miR-9/9*-124) concertedly and separately target components of genetic pathways that antagonize neurogenesis and promote neuronal differentiation during neural development.

The miRNAs capable of converting neurons are capable of converting somatic cells into neurons can coordinate epigenetic and transcriptional changes resulting in neuronal cell fate conversion; induce a generic neuronal state characterized by the loss of fibroblast identity, the presence of a pan-neuronal gene expression program, and absence of subtype specificity; initiate subunit switching within BAF chromatin remodeling complexes while separately repressing the neuronal cell-fate inhibitors REST, Co-REST, and SCP1; or alter the expression of genes involved in DNA methylation, histone modifications, chromatin remodeling, and chromatin compaction.

The miRNAs as described herein can concertedly and separately target components of genetic pathways that antagonize neurogenesis and promote neuronal differentiation during neural development; open the neurogenic potential of adult human fibroblasts and thus provides a platform for subtype-specific neuronal conversion of human cells; orchestrate widespread neuronal chromatin reconfiguration; or promote the opening of neuronal subtype-specific loci, but are not expressed.

A microRNA is a small non-coding RNA molecule (e.g., containing about 22 nucleotides) that can be found in plants, animals, and some viruses, that can function in RNA silencing and post-transcriptional regulation of gene expression. While the majority of miRNAs are located within the cell, some miRNAs, commonly known as circulating miRNAs or extracellular miRNAs, have also been found in an extracellular environment, including various biological fluids and cell culture media.

miRNAs can be encoded by eukaryotic nuclear DNA in plants and animals and by viral DNA in certain viruses whose genome is based on DNA. miRNAs function via base-pairing with complementary sequences within mRNA molecules. As a result, these mRNA molecules are silenced, by one or more of the following processes: cleavage of the mRNA strand into two pieces, destabilization of the mRNA through shortening of its poly(A) tail, or less efficient translation of the mRNA into proteins by ribosomes.

miRNAs can resemble the small interfering RNAs (siRNAs) of the RNA interference (RNAi) pathway, but miRNAs derive from regions of RNA transcripts that fold back on themselves to form short hairpins, whereas siRNAs can derive from longer regions of double-stranded RNA. The human genome can encode over 1000 miRNAs, which are abundant in many mammalian cell types and appear to target about 60% of the genes of humans and other mammals.

MicroRNAs (miRNAs) can regulate genetic pathways by binding to their target transcripts and repressing their expression. Target specificity can be governed largely through short sequence complementarity within the 5′ end of a miRNA enabling a single miRNA to target hundreds of mRNA transcripts. Moreover, a single mRNA can be targeted by multiple miRNAs, markedly enlarging the effect on single gene repression (Wu et al., 2010). These attributes position miRNAs to affect broad changes in gene expression and genetic programs despite their limited size. The convergence of genetic controls by miRNAs towards a specific biological process is exemplified by miR-9/9*- and miRNA-124 miRNAs activated at the onset of neurogenesis. For example, miR-9* and miR-124 can synergistically act as a molecular switch to initiate subunit switching within BAF chromatin remodeling complexes while separately repressing the neuronal cell-fate inhibitors REST, Co-REST, and SCP1. These examples suggest that miR-9/9* and miR-124 target components of genetic pathways that antagonize neurogenesis to promote a neuronal identity during development.

It has been shown that co-expressing the neuronal miRNAs, miR-9/9*-124, with TFs enriched in the cortex and striatum is sufficient to directly convert primary adult human fibroblasts to cortical and striatal medium spiny neurons, respectively. Furthermore, the expression of region-specific TFs alone or substituting miR-9/9*-124 with ASCL1 is insufficient to convert human fibroblasts. Therefore, the use of subtype-specifying TF activity to confer terminal identity during miRNA-mediated neuronal conversion may be reminiscent to in vivo terminal selector TFs which, upon determination of a neuronal fate, initiate and advance mature subtype-identities. However, the existence and utility of such a neuronal state during microRNA-induced neuronal reprogramming has yet to be determined.

In addition to showing the surprising capability of miRNAs, such as miR-9/9*-124, in coordinately stimulating the reconfiguration of chromatin accessibilities, it has been further shown that the microRNA-induced neuronal state enables additional transcription factors, such as ISL1 and LHX3, to selectively commit conversion to a highly homogenous population of human spinal cord motor neurons (Moto-miNs). Furthermore, it has been shown striatal-enriched factors (e.g., CTIP2, DLX1, DLX2 and MYT1L (CDM)) with miR-9/9*-124 generate MSNs from adult human fibroblasts. Taken together, the disclosure reveals a modular synergism between microRNAs and transcription factors that allows lineage-specific neuronal reprogramming, providing a platform for generating distinct subtypes of human neurons.

The transcription factors as described herein can be administered in any method known in the art. For example, transcription factors can be provided exogenously or expressed ectopically.

As described herein, striatal-enriched factors (e.g., CTIP2, DLX1, DLX2, MYT1L (CDM)) with miR-9/9*-124 have been shown to generate MSNs from adult human fibroblasts, yielding a neuronal population comprised of about 70-80% of MSNs.

As described herein, motor neuron factors, ISL1 and LHX3, can function as terminal selectors to specify neuronal conversion to a highly enriched population of human spinal cord motor neurons. Plasticity of the miRNA-induced state was further demonstrated by directly converting adult human fibroblasts into a highly pure population of motor neurons through the addition of motor neuron enriched TFs, ISL1, and LHX3, thereby presenting a modular method to directly convert human fibroblasts into desired neuronal subtypes.

As described herein, neurogenic transcription factors, CTIP2, DLX1, DLX2, and MYT1L (CDM) can reprogram fibroblasts into cortical-like neurons (CN).

In some instances, the transcription factor is a Myt polypeptide. Myt (myelin transcription factor) polypeptides are members of the Myt family of zinc-finger transcription fac-tors. The terms “Myt gene product”, “Myt polypeptide”, and “Myt protein” are used inter-changeably herein to refer to native sequence Myt1 polypeptides, Myt polypeptide variants, Myt polypeptide fragments and chimeric Myt polypeptides that can modulate tran-scription. Native sequence Myt1 polypeptides include the proteins Myt1 (Nzf2; Nztf2; and mKIAA0835; GenBank Accession Nos. NM_008665.3 and NP_032691.2); and MYT1L (myelin transcription factor 1-like; NZF1 Neural zinc finger transcription factor 1; GenBank Accession Nos. NM_015025.2 and NP_055840.2). Myt polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Ac-cession Nos. above find use as reprogramming factors in the present invention, as do nucleic acids encoding these polypeptides or their transcriptionally active domains and vectors comprising these nucleic acids. In certain embodiments, the Myt agent is a MYT1L agent.

In certain embodiments, to a somatic cell (or somatic cell population) are provided the MYT1L transcription factor, miR-9/9*, and miR124. In some embodiments, the somatic cell is provided the MYT1L transcription factor and then about 6 days later the somatic cells are provided miR-9/9*, and miR124.

In some instances, the target non-neuronal somatic cells include or have been modified to include expression cassettes encoding the various components or precursors thereof under the control of an inducible expression system. Any convenient inducible expression system may be employed, where a variety of such systems are known in the art, e.g., the Tet-on inducible expression system. In these instances, the induction agent may be an inducer of the inducible expression system, e.g., tet, dox, etc.

When more than one component makes up the induction agent, e.g., where the induction agent includes two microRNAs and at least one neurogenic factor, the various components may be provided individually or as a single composition, that is, as a premixed composition, of components. The components may be added to the subject cells simultaneously or sequentially at different times. The components may be provided to non-neuronal somatic cells individually or as a single composition, that is, as a premixed composition, of components. The components may be provided at the same molar ratio or at different molar ratios. The components may be provided once or multiple times in the course of culturing the cells of the subject invention. For example, the components may be provided to the subject cells one or more times and the cells allowed to incubate with the components for some amount of time following each contacting event, e.g. 16-24 hours, after which time the media is replaced with fresh media and the cells are cultured further.

For example in a specific embodiment, the MYT1L, miR-9/9*, and miR124 expression vectors are provided to the somatic cells at or about the same time but the miR-9/9*, and miR124 are under control of an inducible promoter (e.g., Tet on system). Thus, MYT1L expression occurs prior to miR-9/9*, and miR124 expression which occurs about 6 days after upon the addition of dox.

In some instances, the cell is contacted with mature versions of the two or more microRNAs and/or TF expression vector(s) of interest under conditions sufficient for the cell to internalize the microRNAs and/or TF expression vector(s). For example, the cell may be contacted with two or more microRNAs in the presence of a transfection agent. Transfection agents of interest include, but are not limited to: Xfect™ transfection reagent from Clontech Laboratories, Lipofectamine LTX transfection reagent from Life Technologies, Lipofectamine 2000 transfection reagent from Life Technologies, SiQuest transfection reagent from Mirus, Transit-siQuest transfection reagent, Transit-TKO transfection reagent, Transit-LTI transfection reagent, Transit-Jurkat transfection reagent, Transit-2020 transfection reagent; chloroquine, PEG, etc. The particular transfection conditions may vary and any convenient protocol may be employed, where suitable protocols are known in the art. Methods for contacting cells with nucleic acid vectors, such as electroporation, calcium chloride transfection, and lipofection, are well known in the art. Vectors that deliver nucleic acids in this manner are usually maintained episomally, e.g. as plasmids or minicircle DNAs.

Instead of contacting the non-neuronal somatic cell with mature forms of the microRNAs of interest, the cell may be contacted with a vector that includes an expression cassette encoding the microRNA of interest or a precursor thereof, e.g., a primary microRNA molecule that can be processed by the cellular machinery of the non-somatic target cell into a pre-microRNA and then ultimately cleaved into the microRNA. Any convenient coding sequence may be employed. For miR-9/9*, coding sequences of interest include, but are not limited to, sequences that encode precursors of miR-9/9* (where both mature microRNAs are generated from the same precursor), e.g., where examples of such coding sequences are reported in mirbase.org. For miR-124, coding sequences of interest include, but are not limited to, sequences that encode precursors of miR-124, e.g., where examples of such coding sequences are reported in mirbase.org. A given vector may include a single coding sequence or multiple repeats of the coding sequence, as desired.

Vectors used for providing microRNA and/or TF expression cassettes to the subject cells may include suitable promoters for driving the expression, that is, transcriptional activation, of the encoding sequence of the expression cassette. This may include ubiquitously acting promoters, for example, the CMV-3-actin promoter, or inducible promoters, such as promoters that are active in particular cell populations or that respond to the presence of drugs such as tetracycline. By transcriptional activation, it is intended that transcription will be increased above basal levels in the target cell by 10-fold or more, by 100-fold or more, such as by 1000-fold or more. In addition, vectors used for providing the nucleic acids may include genes that must later be removed, e.g., using a recombinase system such as Cre/Lox, or the cells that express them destroyed, e.g., by including genes that allow selective toxicity such as herpesvirus TK, bcl-xs, etc

Alternatively, the expression cassette(s) may be provided to the subject cells via a virus. In other words, the cells are contacted with viral particles comprising the expression cassettes. Retroviruses, for example, lentiviruses, are particularly suitable to such methods. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396).

Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject cells are targeted by the packaged viral particles. Suitable methods of introducing the retroviral vectors comprising expression cassettes into packaging cell lines and of collecting the viral particles that are generated by the packaging lines are well known in the art. In those embodiments where the microRNA mediated induction is mediated by three different microRNAs, e.g., miR-9/9*, and miR-124, a single vector may be employed to introduce the expression cassettes of interest or a separate vector may be employed for each expression cassette.

In addition to the TF and/or miRNA agents a given method may include use of other reagents. For example, a given method may include use of one or more agents that promote cell reprogramming. In some embodiments, one or more additional agents include a NEUROD1-activator and/or γ-secretase inhibitor.

In a specific embodiment the NEUROD1-activator is ISX-9 a small molecule shown to act through a calcium-activated signaling pathway dependent on myocyte-enhancer factor 2 (MEF2)-dependent gene expression. In some embodiments, from about PID 3, 6, 10, and/or 14, somatic cells are treated with a NEUROD1-activator, such as ISX9 (e.g., about 10 uM) to push cortical fate.

In a specific embodiment, the γ-secretase inhibitor is DAPT ((2S)—N-[(3,5-Difluorophenyl)acetyl]-L-alanyl-2-phenyl]glycine 1,1-dimethylethyl ester). In some embodiments, from about PID1 to PID14 cells are treated with DAPT (e.g., about 2 uM) to increase neurite outgrowth and/or neuronal differentiation.

Non-limiting examples of other agents known in the art to promote cell reprogramming that may be employed include GSK-3 inhibitors (e.g. CHIR99021 and the like; histone deacetylase (HDAC) inhibitors; histone methyltransferase inhibitors (e.g. G9a histone methyltransferase inhibitors, e.g. BIX-01294, and the like; agonists of the dihydropyridine receptor; and inhibitors of TGF3 signaling (e.g. RepSox and the like. Examples of agents known in the art to promote cell reprogramming also include agents that reduce the amount of methylated DNA in a cell, for example by suppressing DNA methylation activity in the cell or promoting DNA demethylation activity in a cell. Examples of agents that suppress DNA methylation activity include, e.g., agents that inhibit DNA methyltransferases (DNMTs), e.g. 5-aza-cytidine, 5-aza-2′-deoxycytidine, MG98, S-adenosyl-homocysteine (SAH) or an analogue thereof (e.g. periodate-oxidized adenosine or 3-deazaadenosine).

Other reagents of interest for optional inclusion in methods of invention include agents that promote the survival and differentiation of stem cells into neurons and/or mitotic neuronal progenitors or post-mitotic neuronal precursors into neurons. These types of agents include, for example, B27 (Invitrogen), glucose, transferrin, serum (e.g. fetal bovine serum, and the like), and the like. See, e.g. the Examples section presented below.

The various agents of the disclosure (and any optional reagents, as desired), e.g., as described above, may be provided in any convenient culture media, where culture media of interest include those that promote cell survival, e.g. DMEM, Iscoves, Neurobasal media, N3, etc. Media may be supplemented with agents that inhibit the growth of bacterial or yeast, e.g. penicillin/streptomycin, a fungicide, etc., with agents that promote health, e.g. glutamate, and other agents typically provided to culture media as are known in the art of tissue culture.

Non-induction agents of interest, e.g. conversion enhancing agents, agents that promote demethylation, agents that promote the survival and/or differentiation of neurons or subtypes of neurons, agents that inhibit proliferation, and the like, may be provided to the cells prior to providing the induction agent. Alternatively, they may be provided concurrently with providing the induction agent. Alternatively, they may be provided subsequently to providing the induction agent.

In some embodiments, the methods for producing neurons as disclosed herein may include multiple stages (e.g., 2, 3, 4, or more) which include contacting the cells with distinct cell culture mediums and/or transduction steps.

In certain embodiments, the somatic cell is contacted with a one or more culture mediums. The medium may contain any of the following nutrients in appropriate amounts and combinations: salt(s), buffer(s), amino acids, glucose or other sugar(s), antibiotics, serum or serum replacement, and other components such as, but not limited to, apoptosis inhibitor(s), cofactors, and trace elements. In some embodiments, the medium is chemically defined medium.

In some embodiments, the medium comprises a basal medium to which one or more supplements are added. As used herein, a “basal medium” is typically an unsupplemented medium (e.g. Dulbecco's modified Eagle's medium (DMEM), Neurobasal-A). As will be appreciated by those of skill in the art, a basal medium can comprises a variety of components. In some embodiments, the basal medium can further comprise amino acids, salts (e.g., calcium chloride, potassium chloride, magnesium sulfate, sodium chloride, monosodium phosphate), sugars (e.g., glucose), vitamins (e.g., folic acid, nicotinamide, riboflavin, B12), iron, pH indicators (e.g., phenol red), proteins (e.g., albumin), hormones (e.g., insulin), glycoproteins (e.g., transferrin), minerals (e.g., selenium), serum (e.g., fetal bovine serum), antibiotics, antimycotics and glycosaminoglycans.

In one embodiment, a first culture medium for contacting a somatic cell within the first 5-6 days of generating a neuron comprises, consists essentially of, or consists of DMEM, serum, a γ-secretase inhibitor and optionally a NEUROD1-activator. In an exemplary embodiment, from day 1-6 the somatic cells are contacted with a medium comprising DMEM as a basal medium, fetal bovine serum, and DAPT. On about day 3 the NEUROD1-activator (e.g. ISX9) is added to the DMEM as a basal medium, fetal bovine serum, and DAPT medium. Then, the cells contacted with the first medium are contacted with a second medium on about day 6-14 which comprises, consists essentially of, or consists of, Neurobasal-A with B27+ and Glutamax, dibutyl cyclic AMP, valproic acid, DAPT, Ascorbic Acid, BDNF, NT-3, retinoic acid, ISX9, RVC, puromycin and when present, an inducer of miRNA expression (e.g. DOX). After about 14 days, the cells contacted with the second medium can then be cultured in a third medium comprising, consisting essentially of, or consisting of, BrainPhys containing N2 Supplement-A (N2A)(recombinant human insulin, human holo-transferrin (iron-saturated), sodium selenite, putrescine and progesterone) and NeuroCult™ SM1 (SM1)(antioxidants, vitamin A, and insulin), dibutyl cyclic AMP, valproic acid, DAPT, Ascorbic Acid, BDNF, Neurotrophin 3 (NT-3), retinoic acid and when present, an inducer of miRNA expression (e.g. DOX).

In some embodiments, each of the medium compositions can be prepared in a concentrated form. In some embodiments, the medium compositions can be prepared in a concentrated form suitable for dilution. In some embodiments, medium compositions prepared in a concentrated form can be suitable for at least about 1×, about 2×, about 3×, about 4×, about 5×, about 6×, about 7×, about 8×, about 9×, or about 10× dilution.

In some embodiments, the somatic cells used in the in vitro culturing system disclosed herein for producing neuron cells may be genetically modified, and/or the resultant neurons genetically modified. Accordingly, the present disclosure also provides methods of preparing such genetically neuronal cells according to the present disclosure (see the below examples). In some embodiments, the cells are genetically modified to comprise a disrupted gene. As used herein, the term “a disrupted gene” refers to a gene containing one or more mutations (e.g., insertion, deletion, or nucleotide substitution, etc.) relative to the wild-type counterpart (e.g. healthy control) so as to substantially modulate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or express a substantially reduced level of the encoded protein. In other instances, the disrupted gene expresses the encoded protein in a mutated form, which is either not functional, has substantially reduced activity, or increased activity. In some embodiments, a disrupted gene does not express (e.g., encode) a functional protein. In other embodiments, the genetically modified cell incorporates a heterologous nucleic acid. The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

In addition, techniques such as CRISPR (particularly using Cas9 and guide RNA), editing with zinc finger nucleases (ZFNs) and transcription activator-like effector nucleases (TALENs) may be used to produce the genetically engineered pluripotent stem cells.

‘Genetic modification’, ‘genome editing’, or ‘genomic editing’, or ‘genetic editing’, as used interchangeably herein, is a type of genetic engineering in which DNA is inserted, deleted, and/or replaced in the genome of a targeted cell. Targeted genome modification (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. In another aspect, an endogenous gene may be modified by introducing a change in an endogenous gene codon, wherein the modification introduces an amino acid change in the gene product or introduction of a stop codon. Therefore, targeted modification may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted modification can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted modification could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

In some embodiments, non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

In an exemplary embodiment, the CRISPR/Cas9 gene editing technology is used for producing the genetically engineered pluripotent stem cells. Typically, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. Any known CRISPR/Cas9 methods can be used in the methods disclosed herein. See also Examples below.

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wp/SPBc/TP901-1, whether used individually or in combination.

Any of the gene editing nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

Tauopathy is a pathogenic process that underlies many forms of neurodegenerative disorders including Alzheimer's disease and primary tauopathy. A key requirement to properly study the adult-onset tauopathy is the ability to replicate the expression of tau isoforms typically seen in adult human brains. Whether one can generate human neurons that express the 4 repeat (4R) tau isoform, in particular, has been a question of paramount importance, since adult human brain forms the 1:1 ratio between 3R and 4R tau isoforms and perturbation of this ratio has been implicated in the onset of tau aggregation and tauopathy. So far, this has remained a challenge as neurons differentiated from induced pluripotent stem cells (iPSCs) express primarily 3R tau as iPSC-derived neurons reflect the fetal age, and there has been no demonstration of reprogrammed human neurons that precisely mirror the 3R and 4R ratio at the level similar to the human adult brain. Using microRNA (miRNA)-based reprogramming of human adult fibroblasts to neurons, it is shown here that miRNA-induced neurons robustly recapitulate the expression of all six tau isoforms expressed in adult brains, with 4R tau establishing the 1:1 ratio with 3R tau. The reprogramming protocol leverages the activity of miR-9/9* and miR-124 (miR-9/9*-124) as cell reprogramming agents that my lab has pioneered and combines it with a small molecule ISX-9 (ISX9) to guide the neuronal conversion to cortical neurons. The resulting reprogrammed neurons express endogenously 3R and 4R-tau levels analogous to the human adult brain. We applied this finding to samples from familial tauopathy patients who contain a mutation within the tau gene that affects tau splicing and increases 4R tau, and found that the patient-derived neurons show disrupted 3R:4R balance leading to tau aggregation. This microRNA-induced neurons, thus, serve as a robust platform to track 4R tau expression and identity genes or small molecules that could modulate this process.

### II. In Vitro Cell Culturing Systems and Uses Thereof

Any of the neuron cells produced by the methods of various aspects described herein (e.g., the methods of Section I) can be used in different applications where neurons are required. Such neuronal cells are also within the scope of the present disclosure. In some embodiments, the neuron cells expression of all six tau isoforms expressed in adult brains, with 4R tau establishing the 1:1 ratio with 3R tau (when starting from a healthy (i.e. unmutated) somatic cell source).

In some aspect, provided herein is an in vitro cell culture system, which comprises a cell culture vessel. In one aspect, the in vitro cell culture system comprises neurons generated from a population of somatic cells, wherein the somatic cells or resultant neurons are genetically modified. Suitable cell culture vessels are not particularly limited and can include any single or multi-well vessel.

Any of the in vitro cell culture system disclosed herein can be used, for example, to advance therapeutic discovery. Accordingly, provided herein include a method of screening for an agent for useful for treating a tauopathy or CNS disease is also provided herein. The method generally comprises providing an in vitro cell culture system as disclosed herein and culturing the neuron cells in the presence of a candidate agent and optionally measuring tau precipitation, tau expression, cell viability, and/or cell health. In some embodiments, the candidate agent is identified the candidate agent as an agent for treating a tauopathy or CNS disease if the candidate agent improves or maintains the neuronal cells to a healthy control like state.

The candidate agents can be selected from the group consisting of proteins, peptides, nucleic acids (e.g., but not limited to, siRNA, anti-miRs, antisense oligonucleotides, and ribozymes), small molecules, nutrients (lipid precursors), and a combination of two or more thereof.

In some embodiments, effects of the candidate agents on the neuron cells of the disclosure can be determined by measuring response of the cells and comparing the measured response with neuron cells that are not contacted with the candidate agents. Various methods to measure cell response are known in the art, including, but not limited to, cell labeling, immunostaining, optical or microscopic imaging {e.g., immunofluorescence microscopy and/or scanning electron microscopy), spectroscopy, gene expression analysis, cytokine/chemokine secretion analysis, metabolite analysis, polymerase chain reaction (PCR), immunoassays, ELISA, gene arrays, spectroscopy, immunostaining, electrochemical detection, polynucleotide detection, fluorescence anisotropy, fluorescence resonance energy transfer, electron transfer, enzyme assay, magnetism, electrical conductivity (e.g., trans-epithelial electrical resistance (TEER)), isoelectric focusing, chromatography, immunoprecipitation, immunoseparation, aptamer binding, filtration, electrophoresis, use of a CCD camera, mass spectroscopy, or any combination thereof. Detection, such as cell detection, can be carried out using light microscopy with phase contrast imaging and/or fluorescence microscopy based on the characteristic size, shape and refractile characteristics of specific cell types.

The present disclosure provides for converted neurons and uses thereof for models of disease using a patient's fibroblast cells, ectopic expression of microRNAs, and transcription factors (see e.g., Example 2).

As described herein, the identification of miRNA-induced neurogenic state has provided molecular insights into how multiple neuronal subtypes can be generated from patient fibroblasts for modeling neurological diseases. For example, the methods described herein can be used to convert a somatic cell such as a fibroblast cell (e.g., human fibroblast cells) into a converted neuron. The somatic cell or fibroblast cell can be any somatic cell or fibroblast capable of being converted using any of the methods as described herein. The converted neuron can be a microRNA-induced neuron (miN). For example, the converted neuron can be a motor neuron, a spinal motor neuron, a cortical neuron, a cortical-like neuron, a striatal medium spiny neuron (MSN), a dopaminergic neuron, a GABAergic neuron, a cholinergic neuron, serotonergic neuron, or a glutamatergic neuron.

Ectopic expression of brain-enriched microRNAs (miRNAs), such as miR-9/9* and miR-124 (miR-9/9*-124), in human adult fibroblasts, have been shown to directly convert fibroblasts to neurons. The miR-9/9*-124-mediated conversion, partially afforded by their activity in controlling chromatin remodeling complexes, can be guided to specific and mature neuronal subtypes with the co-expression of transcription factors. As such, striatal factors (or striatal-enriched factors) CTIP2, DLX1, DLX2, and MYT1L (CDM) with miR-9/9*-124 have been shown to generate MSNs from adult human fibroblasts, yielding a neuronal population comprised of 70-80% of MSNs. Given that iPSC-based protocols have reported MSN conversion efficiencies of only 5%-10% and more recently 20-40%, the MSN-specific neuronal conversion that generates a neuronal population highly enriched with MSNs from HD patients will offer a useful tool to model HD. Moreover, in contrast to neurons differentiated from iPSCs in which the age stored in original fibroblasts is erased during the induction of pluripotency, directly converted neurons has been shown to retain age-associated marks of starting adult human fibroblasts, including the epigenetic age (also known as the epigenetic clock), oxidative stress, DNA damage, miRNAome, telomere lengths and transcriptome. This unique feature offers potential advantages in modeling adult-onset disorders using directly converted neurons, yet the value of MSNs converted from HD patients' fibroblasts in disease modeling has not been determined.

The generation of HD patient-derived MSNs (HD-MSNs) through miR-9/9*-124-CDM-based conversion of fibroblasts is reported herein. The present disclosure focused on HD samples with CAG repeat ranges in the 40 s, which represent the majority of HD cases, in contrast to previously reported studies on modeling HD with CAG repeat numbers longer than 60 CAG repeats. It was found that HD-MSNs captured many HD-associated phenotypes, including formation of aberrant protein aggregates, mHTT-induced DNA damage, spontaneous degeneration over time in culture, and decline in mitochondrial function. Furthermore, by inducing HD fibroblasts into iPSCs and redifferentiating them back into fibroblasts for miRNA-based neuronal conversion, it was discovered that differences in mHTT aggregation propensity observed in these two distinct cellular reprogramming methods are the result of drastically different levels of proteasome activity. Intriguingly, modifying the terminal neuronal cell fate to cortical neurons in directly reprogrammed HD cells alleviated mHTT-induced toxicity through reduced DNA damage and reduced cell death. Furthermore, MSNs reprogrammed from six pre-symptomatic HD patients, sampled at least 13 years before the clinical onset of the disease, were less vulnerable to mHTT-induced toxicity despite the marked presence of mHTT aggregates. These data highlight the advantages of direct neuronal conversion offers for modeling age-related phenotypes of late-onset diseases with enriched populations of specific neuronal subtypes. While the applicability of iPSCs for the development of stem cell-based therapies and modeling of developmental processes remains unequivocal, the present findings address many of the challenges for modeling adult-onset HD.

In Huntington's disease (HD), expansion of CAG codons within the Huntington gene (HTT) leads to the aberrant formation of protein aggregates and the differential degeneration of striatal medium spiny neurons (MSNs). Modeling HD using patient-specific MSNs has been challenging, as neurons differentiated from induced pluripotent stem cells are free of aggregates and lack an overt cell death phenotype. Here MSNs from HD patient fibroblasts were generated through microRNA-based neuronal conversion, which has previously been shown to bypass the induction of pluripotency and retain age signatures of original fibroblasts. It was found that patient MSNs consistently exhibited mutant HTT (mHTT) aggregates, and spontaneous degeneration over time in culture that was preceded by mHTT-dependent DNA damage. Further evidence is provided that erasure of age stored in starting fibroblasts or diverting conversion into cortical neurons resulted in differential manifestation of cellular phenotypes associated with HD, highlighting the importance of age and neuronal subtype specificity in modeling late-onset neurological disorders.

Huntington's disease (HD) is a progressive neurodegenerative disorder caused by the abnormal expansion of CAG codons within the first exon of the Huntington (HTT) gene. HD symptoms typically manifest in midlife, and include motor deficits, psychiatric symptoms and cognitive decline. While healthy individuals have an average HTT CAG tract size of 17-20 repeats, HD patients have an expansion of 36 or more CAGs4. Moreover, CAG repeat length is directly correlated to severity of the disease and inversely related to age of onset, with abnormally large CAG expansions (>60 repeats) leading to juvenile onset. Expanded CAG trinucleotides encode a polyglutamine stretch (PolyQ) that can accumulate into proteinaceous cytoplasmic and intranuclear aggregates that are generally thought to be neurotoxic, although the formation of inclusion bodies has also been suggested as a neuroprotective mechanism.

A striking characteristic of HD pathology is the selective degeneration of striatal medium spiny neurons (MSNs), while other neuronal subpopulations are relatively spared. Due to the clinical importance of MSNs and the lack of treatment capable of halting HD onset and progression, many protocols have been developed to generate MSNs from induced pluripotent stem cells (iPSCs) to establish patient-specific platforms for disease modeling.

Studies modeling HD with patient-specific iPSC-derived MSNs have, however, only uncovered mild phenotypes, often requiring additional cellular insults. For example, striatal neurons generated through HD-iPSCs demonstrated elevated levels of caspase activity only upon trophic factor withdrawal, treatment with hydrogen peroxide or high levels of glutamate, but otherwise displayed no overt cell death phenotype. In addition, neurons differentiated from iPSCs of HD patients did not display mutant HTT (mHTT) aggregates even after the addition of cellular stressors, and in other studies required culturing for at least 6-8 months and treatment with proteasome inhibitors before aggregates were detected. Therefore, a different reprogramming approach that generates a homogenous population of patient-derived MSNs that display HD phenotypes more robustly will offer an alternative cellular platform for disease modeling and drug screening.

The present disclosure provides for methods and compositions for treating or modeling neurodegenerative (e.g., neurological, motor neuron, tauopathy) diseases, disorders, or conditions or screening for therapeutics for neurological diseases, disorders, or conditions. For example, a tauopathy can be a heterogeneous group of neurodegenerative diseases characterized by abnormal metabolism of misfolded T (tau) proteins leading to intracellular accumulation and formation of neurofibrillary tangles (NFT). These neurofibrillary tangles are deposited in the cytosol of neurons and glial cells. Examples of tauopathies can include progressive supranuclear palsy (PSP); frontotemporal lobar degeneration (FTLD-TAU); corticobasal degeneration; or Alzheimer's disease (considered a secondary tauopathy). As another example, a neurodegenerative disease, disorder, or condition can be Abulia; Agraphia; Alcoholism; Alexia; Alien hand syndrome; Allan-Herndon-Dudley syndrome; Alternating hemiplegia of childhood; Alzheimer's Disease (AD); Amaurosis fugax; Amnesia; Amyotrophic lateral sclerosis (ALS); Aneurysm; Angelman syndrome; Anosognosia; Aphasia; Apraxia; Arachnoiditis; Arnold-Chiari malformation; Asomatognosia; Asperger syndrome; Ataxia; Attention deficit hyperactivity disorder; ATR-16 syndrome; Auditory processing disorder; Autism spectrum; Behcets disease; Bipolar disorder; Bell's palsy; Brachial plexus injury; Brain damage; Brain injury; Brain tumor; Brody myopathy; Canavan disease; Capgras delusion; Carpal tunnel syndrome; Causalgia; Central pain syndrome; Central pontine myelinolysis; Centronuclear myopathy; Cephalic disorder; Cerebral aneurysm; Cerebral arteriosclerosis; Cerebral atrophy; Cerebral autosomal dominant arteriopathy with subcortical infarcts and leukoencephalopathy (CADASIL); Cerebral dysgenesis-neuropathy-ichthyosis-keratoderma syndrome (CEDNIK syndrome); Cerebral gigantism; Cerebral palsy; Cerebral vasculitis; Cervical spinal stenosis; Charcot-Marie-Tooth disease; Chiari malformation; Chorea; Chronic fatigue syndrome; Chronic inflammatory demyelinating polyneuropathy (CIDP); Chronic pain; Cockayne syndrome; Coffin-Lowry syndrome; Coma; Complex regional pain syndrome; Compression neuropathy; Congenital facial diplegia; Corticobasal degeneration; Cranial arteritis; Craniosynostosis; Creutzfeldt-Jakob disease; Cumulative trauma disorders; Cushing's syndrome; Cyclothymic disorder; Cyclic Vomiting Syndrome (CVS); Cytomegalic inclusion body disease (CIBD); Cytomegalovirus Infection; Dandy-Walker syndrome; Dawson disease; De Morsier's syndrome; Dejerine-Klumpke palsy; Dejerine-Sottas disease; Delayed sleep phase syndrome; Dementia; Dermatomyositis; Developmental coordination disorder; Diabetic neuropathy; Diffuse sclerosis; Diplopia; Disorders of consciousness; Down syndrome; Dravet syndrome; Duchenne muscular dystrophy; Dysarthria; Dysautonomia; Dyscalculia; Dysgraphia; Dyskinesia; Dyslexia; Dystonia; Empty sella syndrome; Encephalitis; Encephalocele; Encephalotrigeminal angiomatosis; Encopresis; Enuresis; Epilepsy; Epilepsy-intellectual disability in females; Erb's palsy; Erythromelalgia; Essential tremor; Exploding head syndrome; Fabry's disease; Fahr's syndrome; Fainting; Familial spastic paralysis; Febrile seizures; Fisher syndrome; Friedreich's ataxia; Fibromyalgia; Foville's syndrome; Fetal alcohol syndrome; Fragile X syndrome; Fragile X-associated tremor/ataxia syndrome (FXTAS); Gaucher's disease; Generalized epilepsy with febrile seizures plus; Gerstmann's syndrome; Giant cell arteritis; Giant cell inclusion disease; Globoid Cell Leukodystrophy; Gray matter heterotopia; Guillain-Barré syndrome; Generalized anxiety disorder; HTLV-1 associated myelopathy; Hallervorden-Spatz syndrome; Head injury; Headache; Hemifacial Spasm; Hereditary Spastic Paraplegia; Heredopathia atactica polyneuritiformis; Herpes zoster oticus; Herpes zoster; Hirayama syndrome; Hirschsprung's disease; Holmes-Adie syndrome; Holoprosencephaly; Huntington's disease; Hydranencephaly; Hydrocephalus; Hypercortisolism; Hypoxia; Immune-Mediated encephalomyelitis; Inclusion body myositis; Incontinentia pigmenti; Infantile Refsum disease; Infantile spasms; Inflammatory myopathy; Intracranial cyst; Intracranial hypertension; Isodicentric 15; Joubert syndrome; Karak syndrome; Kearns-Sayre syndrome; Kinsbourne syndrome; Kleine-Levin syndrome; Klippel Feil syndrome; Krabbe disease; Kufor-Rakeb syndrome; Lafora disease; Lambert-Eaton myasthenic syndrome; Landau-Kleffner syndrome; Lateral medullary (Wallenberg) syndrome; Learning disabilities; Leigh's disease; Lennox-Gastaut syndrome; Lesch-Nyhan syndrome; Leukodystrophy; Leukoencephalopathy with vanishing white matter; Lewy body dementia; Lissencephaly; Locked-in syndrome; Lou Gehrig's disease (See amyotrophic lateral sclerosis); Lumbar disc disease; Lumbar spinal stenosis; Lyme disease—Neurological Sequelae; Machado-Joseph disease (Spinocerebellar ataxia type 3); Macrencephaly; Macropsia; Mal de debarquement; Megalencephalic leukoencephalopathy with subcortical cysts; Megalencephaly; Melkersson-Rosenthal syndrome; Menieres disease; Meningitis; Menkes disease; Metachromatic leukodystrophy; Microcephaly; Micropsia; Migraine; Miller Fisher syndrome; Mini-stroke (transient ischemic attack); Misophonia; Mitochondrial myopathy; Mobius syndrome; Monomelic amyotrophy; Morvan syndrome; Motor Neurone Disease—see amyotrophic lateral sclerosis; Motor skills disorder; Moyamoya disease; Mucopolysaccharidoses; Multi-infarct dementia; Multifocal motor neuropathy; Multiple sclerosis; Multiple system atrophy; Muscular dystrophy; Myalgic encephalomyelitis; Myasthenia gravis; Myelinoclastic diffuse sclerosis; Myoclonic Encephalopathy of infants; Myoclonus; Myopathy; Myotubular myopathy; Myotonia congenita; Narcolepsy; Neuro-Behçet's disease; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Neuropathy; Neurosis; Niemann-Pick disease; Non-24-hour sleep-wake disorder; Nonverbal learning disorder; O'Sullivan-McLeod syndrome; Occipital Neuralgia; Occult Spinal Dysraphism Sequence; Ohtahara syndrome; Olivopontocerebellar atrophy; Opsoclonus myoclonus syndrome; Optic neuritis; Orthostatic Hypotension; Otosclerosis; Overuse syndrome; Palinopsia; Paresthesia; Parkinson's disease; Paramyotonia congenita; Paraneoplastic diseases; Paroxysmal attacks; Parry-Romberg syndrome; PANDAS; Pelizaeus-Merzbacher disease; Periodic paralyses; Peripheral neuropathy; Pervasive developmental disorders; Phantom limb/Phantom pain; Photic sneeze reflex; Phytanic acid storage disease; Pick's disease; Pinched nerve; Pituitary tumors; PMG; Polyneuropathy; Polio; Polymicrogyria; Polymyositis; Porencephaly; Post-polio syndrome; Postherpetic neuralgia (PHN); Postural hypotension; Prader-Willi syndrome; Primary lateral sclerosis; Prion diseases; Progressive hemifacial atrophy; Progressive multifocal leukoencephalopathy; Progressive supranuclear palsy; Prosopagnosia; Pseudotumor cerebri; Quadrantanopia; Quadriplegia; Rabies; Radiculopathy; Ramsay Hunt syndrome type I; Ramsay Hunt syndrome type II; Ramsay Hunt syndrome type III—see Ramsay-Hunt syndrome; Rasmussen encephalitis; Reflex neurovascular dystrophy; Refsum disease; REM sleep behavior disorder; Repetitive stress injury; Restless legs syndrome; Retrovirus-associated myelopathy; Rett syndrome; Reye's syndrome; Rhythmic Movement Disorder; Romberg syndrome; Saint Vitus dance; Sandhoff disease; Schilder's disease (two distinct conditions); Schizencephaly; Sensory processing disorder; Septo-optic dysplasia; Shaken baby syndrome; Shingles; Shy-Drager syndrome; Sjögren's syndrome; Sleep apnea; Sleeping sickness; Snatiation; Sotos syndrome; Spasticity; Spina bifida; Spinal cord injury (SCI); Spinal cord tumors; Spinal muscular atrophy; Spinal and bulbar muscular atrophy; Spinocerebellar ataxia; Split-brain; Steele-Richardson-Olszewski syndrome; Stiff-person syndrome; Stroke; Sturge-Weber syndrome; Stuttering; Subacute sclerosing panencephalitis; Subcortical arteriosclerotic encephalopathy; Superficial siderosis; Sydenham's chorea; Syncope; Synesthesia; Syringomyelia; Tarsal tunnel syndrome; Tardive dyskinesia; Tardive dysphrenia; Tarlov cyst; Tay-Sachs disease; Temporal arteritis; Temporal lobe epilepsy; Tetanus; Tethered spinal cord syndrome; Thomsen disease; Thoracic outlet syndrome; Tic Douloureux; Todd's paralysis; Tourette syndrome; Toxic encephalopathy; Transient ischemic attack; Transmissible spongiform encephalopathies; Transverse myelitis; Traumatic brain injury; Tremor; Trichotillomania; Trigeminal neuralgia; Tropical spastic paraparesis; Trypanosomiasis; Tuberous sclerosis; 22q13 deletion syndrome; Unverricht-Lundborg disease; Vestibular schwannoma (Acoustic neuroma); Von Hippel-Lindau disease (VHL); Viliuisk Encephalomyelitis (VE); Wallenberg's syndrome; West syndrome; Whiplash; Williams syndrome; Wilson's disease; Y-Linked Hearing Impairment; or Zellweger syndrome.

Taupathy: Tauopathies are a heterogeneous group of neurodegenerative diseases characterized by abnormal metabolism of misfolded T (tau) proteins leading to intracellular accumulation and formation of neurofibrillary tangles (NFT). These neurofibrillary tangles are deposited in the cytosol of neurons and glial cells. Examples of tauopathies can include progressive supranuclear palsy (PSP); frontotemporal lobar degeneration (FTLD-TAU); corticobasal degeneration; or Alzheimer's disease (considered a secondary tauopathy).

Some texts define tauopathies as a disease characterized by mutations in the T protein gene itself. If such a strict definition is used, even though the histopathological hallmark of Alzheimer's disease is the presence of numerous neurofibrillary tangles (which are also formed by T proteins), it may not be strictly considered a tauopathy, as no defect in the tau protein gene has been identified. Thus, it can be referred to as a secondary tauopathy as β-amyloid accumulation is considered the primary pathology. Here, a taupathy can be a primary or secondary taupathy. Tauopathies are the result of aggregation and precipitation of misfolded T proteins that normally stabilize neural microtubules. These aggregates form neurofibrillary tangles that in turn lead to neuronal toxicity and degeneration. Misfolded T proteins from two distinct aggregates: three repeat (3R) and four repeat (4R) which variably present in different diseases. More recently the discovery of the glymphatic pathway and the importance of this in the normal physiological clearance of extracellular solutes including beta-amyloid, suggests that there is also the possibility of reduced clearance, in addition to abnormal metabolism, as the underpinning of some tauopathies including chronic traumatic encephalopathy.

Motor Neuron Disease: As described herein, the present disclosure provides for the treatment of a neurodegenerative disease (e.g., a motor neuron disease) using a converted neuron, by expression of miR-9/9* and miR-124 (miR-9/9*-124) and transcription factors (i.e., ISL1 and LHX3) in a human adult fibroblast.

In some embodiments, the neurodegenerative disease, disorder or condition can be a motor neuron disease (MND). Motor neuron diseases (MNDs) are a group of progressive neurological disorders that can destroy motor neurons, the cells that control essential voluntary muscle activity such as speaking, walking, breathing, and swallowing. A motor neuron disease can be an inherited disease with symptoms including difficulty or inability to grip, walk, speak, swallow, or breathe; a weakened grip, which can cause difficulty picking up or holding objects; weakness at the shoulder that makes lifting the arm difficult; a “foot drop” caused by weak ankle muscles; dragging of the leg; or slurred speech (dysarthria).

As another example, a motor neuron disease can be Amyotrophic Lateral Sclerosis (ALS), Spinal Muscular Atrophy (SMA), or Spinal Cord Injury (SCI). Other motor neuron diseases can include frontotemporal dementia, progressive bulbar palsy, pseudobulbar palsy, primary lateral sclerosis (PLS), progressive muscular atrophy, Spinal muscular atrophy (SMA) (e.g., SMA type I, also called Werdnig-Hoffmann disease; SMA type II; congenital SMA with arthrogryposis; Kennedy's disease, also known as progressive spinobulbar muscular atrophy), or post-polio syndrome (PPS).

Also provided are screening methods. As described herein, these reprogrammed neurons can be used as a cellular platform expressing tau isoforms of adult brains, which can be used to identify compounds or genes that can modulate it.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to somatic cells, fibroblasts, ISX9 or other NEUROD1 activator, transcription factors, plasmids, expression vectors, or combinations thereof. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, the term “consisting essentially of” takes it's well established meaning and is generally construed to mean that the composition or formulation (a) necessarily includes the listed ingredients and (b) is open to unlisted ingredients that do not materially affect the basic and novel properties of the composition (e.g. resulting in high-recovery yields of cryopreserved SC-islets; improved islet health resulting in improved function; high-recovery yield of resized SC-islets; improved cell recovery and clustering morphology; improved gene expression for SC Islets and maintains Beta cell identity; maintenance of C-peptide and NKX6-1; increase in INS or MAFA gene expression; or maintain insulin expression after thaw relative to standard techniques).

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Recapitulation of Endogenous 4R Tau Expression and Formation of Insoluble Tau in Directly Reprogrammed Human Neurons

Tau is a microtubule-binding protein expressed in neurons and the equal ratio between 4-repeat (4R) and 3-repeat (3R) isoforms are maintained in normal adult brain function. Dysregulation of 3R:4R ratio causes tauopathy and human neurons that recapitulate tau isoforms in health and disease will provide a platform for elucidating pathogenic processes involving tau pathology. The present example carried out extensive characterizations of tau isoforms expressed in human neurons derived by microRNA-induced neuronal reprogramming of adult fibroblasts. Transcript and protein analyses showed miR-neurons expressed all six isoforms with the 3R:4R isoform ratio equivalent to that detected in human adult brains. Also, miR-neurons derived from familial tauopathy patients with a 3R:4R ratio altering mutation showed increased 4R tau and the formation of insoluble tau with seeding activity. The present example provides the utility of miRNA-induced neuronal reprogramming to recapitulate endogenous tau regulation comparable to the adult brain in health and disease.

### Results

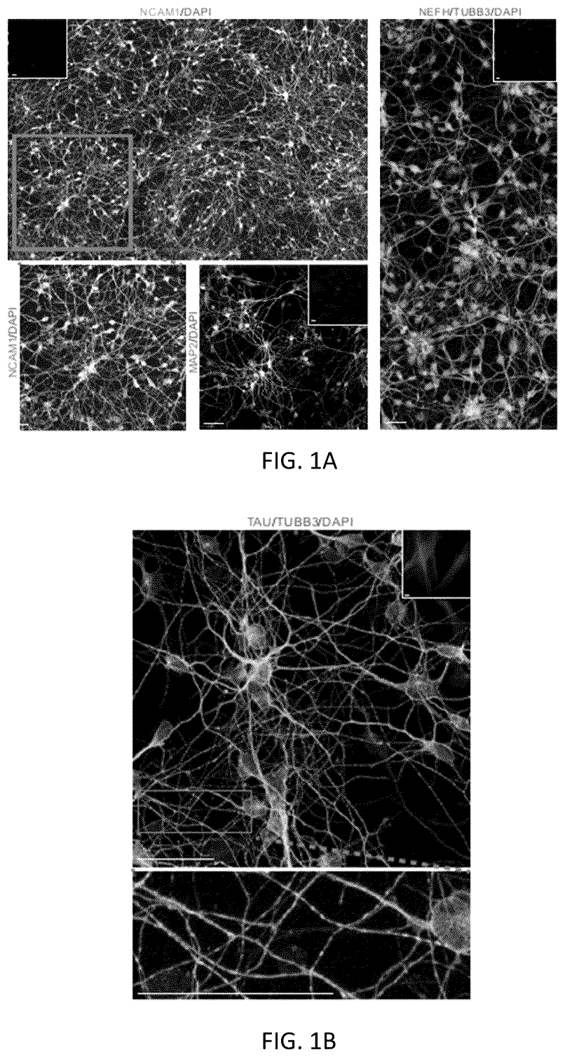

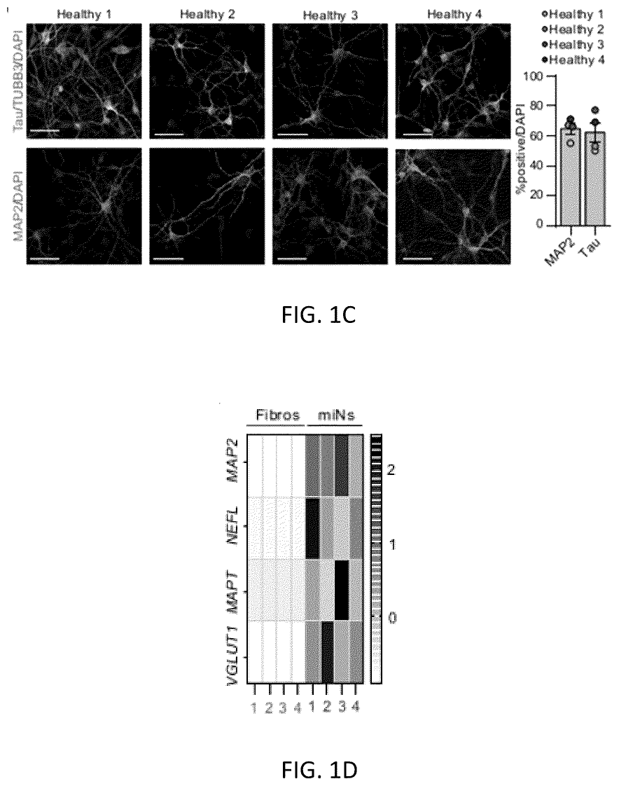

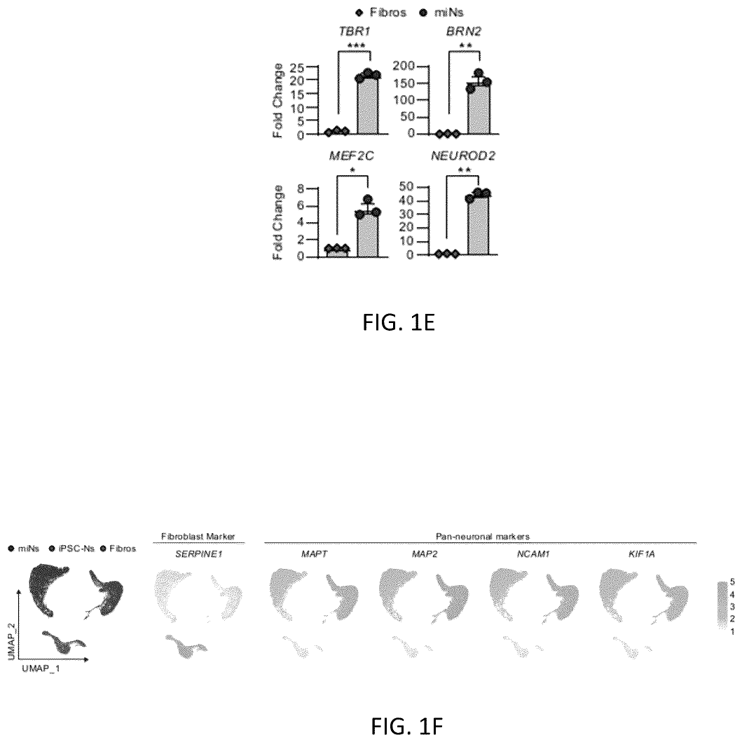

MiNs display neuronal markers similar to iPSC-Ns and fibroblast fate erasure: The neuronal conversion protocol based on miR-9/9*-124, Neuronal Differentiation 2 (NEUROD2), and Myelin Transcription Factor 1 Like (MYT1L) transcription factors (TFs) was adapted to generate neurons of cortical lineage (FIG. 1). To minimize the number of lentiviruses used for transduction in fibroblasts, NEUROD2 was replaced with ISX9, a neurogenic compound shown to activate endogenous NEUROD-family TFs.

At thirty days post-induction (PID 30), immunostaining for pan-neuronal marker Neuronal Cell Adhesion Molecule 1 (NCAM1) showed a robust reprogramming output with miNs displaying extensive process outgrowths (FIG. 1A left, bottom-left: zoomed in view of NCAM1 expression). The reprogrammed cells were also enriched for other pan-neuronal markers, MAP2 (Microtubule Associated Protein 2) (FIG. 1A, bottom-right) and Neurofilament Heavy Chain (FIG. 1A, right). Beta III tubulin (TUBB3) was used to monitor cell morphologies. Importantly, immunostaining against tau revealed the miN population highly enriched with tau expression showing prototypical patterns of tau islands in TUBB3-positive outer neurites (FIG. 1B), an endogenous tau feature suggested to be reflective of tau-tau interactions.

Using immunocytochemistry on four independent fibroblast samples reprogrammed into miNs (FIG. 1C), approximately 60-70% of DAPI-positive cells showed MAP2- and tau-positivity, which is consistent with the reprogramming efficiency previously reported. qPCR analyses of neuronal genes, MAP2, NEFL (Neurofilament Light Chain), MAPT, and the glutamatergic marker, SLC17A7 (also known as VGLUT1), showed enrichment over starting fibroblasts (FIG. 1D). Also, qPCR analyses of the cortical markers TBR1 (T-Box Brain Transcription Factor 1), MEF2C (Myocyte Enhancer Factor 2C), BRN2 (POU Class 3 Homeobox 2), and NEUROD2 demonstrated that the combination of MYT1L and ISX9 are sufficient to drive the cortical fate (FIG. 1E).

Using single-cell RNA-sequencing (scRNA-seq) to stratify starting fibroblasts (n=2533), reprogrammed miNs (n=9305), and iPSC-Ns (n=7212) by their transcriptome, three distinct cell populations were identified corresponding to fibroblasts, miNs, and iPSC-Ns (FIG. 1F, left). For instance, the non-neuronal marker SERPINE1 was enriched only in starting fibroblasts, whereas the miNs and iPSC-Ns demonstrated thorough expression of pan-neuronal markers, MAPT, MAP2, NCAM1, and KIF1A (FIG. 1F, right). Additionally, miNs were characterized by high expression of SLC17A7 (VGLUT1) whereas iPSC-Ns were positive for SLC17A6 (VGLUT2). MiNs and iPSC-Ns expressed different glutamate receptor subunits, but both were highly enriched for the glutamine synthetase, GLUL. GAD1 and GAD2 were present in a subset of miNs and iPSC-Ns, respectively. However, the expression of GABA transporter, SLC6A1, was only marginal. MiNs showed a low number of PVALB-positive cells, whereas iPSC-Ns had small populations of SST and TH positive cells. Together, these results support the notion that miNs robustly acquire a neuronal fate due to miRNA-mediated reprogramming.

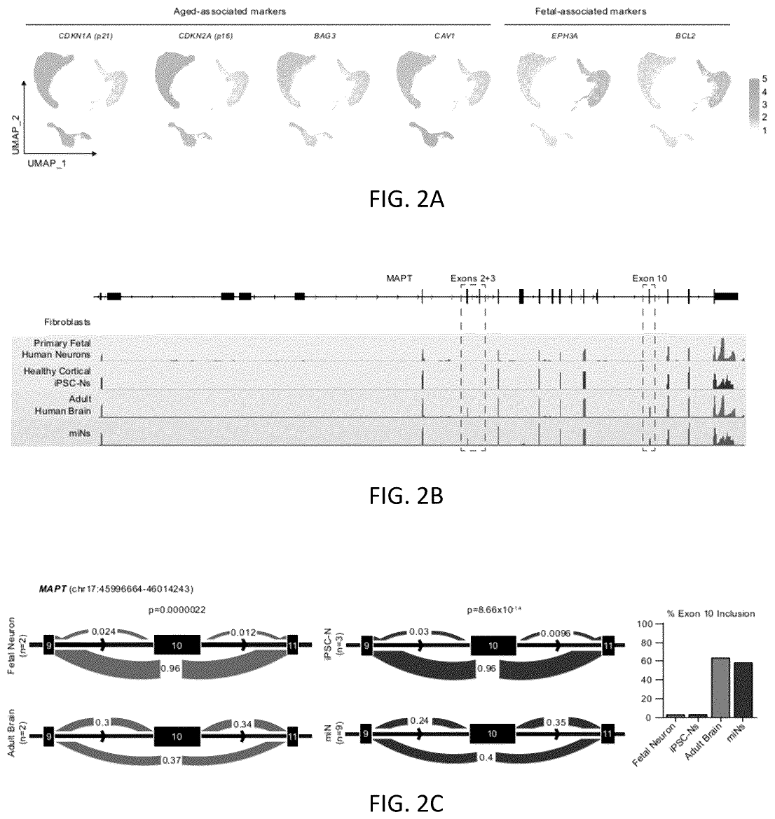

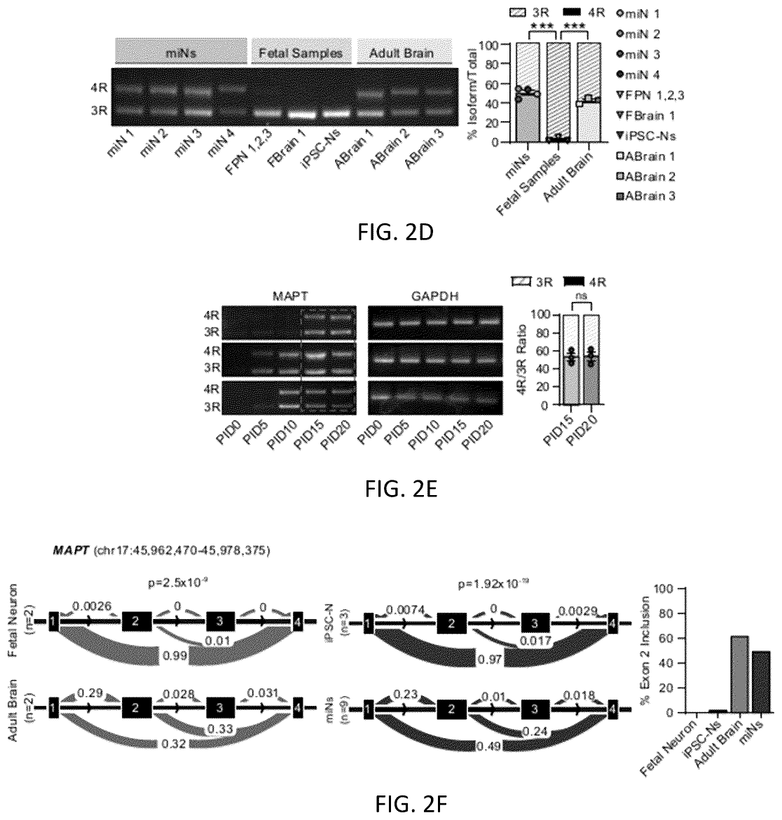

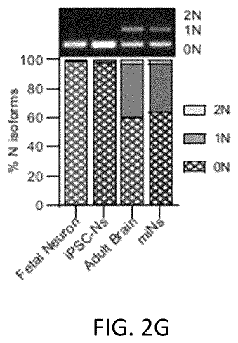

Age-maintained miNs endogenously express a 1:1 3R:4R tau isoform ratio equivalent to adult brain: scRNA-seq revealed that age-associated markers as main drivers for distinct segregation of miNs and iPSC-Ns. For instance, miNs were positive for the advanced age markers CDKN1A (p21), CDKN2A (p16), BAG3, and CAV1 (FIG. 2A). BAG3 expression dramatically increases with aging in mouse neurons and the human brain (brainspan.org), and has been implicated in tau clearance. Moreover, the expression of CAV1 has been shown to be increased in aged human brains and its abnormal expression has been implicated in Parkinson's Disease. In contrast, fetal-associated markers such as EPH3A (brainspan.org) and BCL2 were enriched in iPSC-Ns. These results conform to previous studies indicating the maintenance of cellular age in direct neuronal reprogramming.

Tau isoform expression is developmentally regulated in which 4R isoforms begin to be expressed perinatally and are maintained throughout adulthood. Extending the notion of age maintenance, if miNs expressed 4R tau was tested by bulk RNA-sequencing (RNAseq). Strikingly, miNs and adult brain showed robust signal of exon 10 in the RNAseq tracks (FIG. 2B, red box: blue and green tracks, respectively), peaks that were distinctly missing in primary fetal human neurons and iPSC-Ns (FIG. 2B, red dashed box: grey and purple tracks, respectively). The alternatively spliced exons 2 and 3 are also clearly expressed in miNs and adult human brain (FIG. 2B, pink dashed box). Leafcutter analysis was also used, which calculates splicing events, to quantify inclusion of exon 10 and found significant differences between fetal and adult brain (p=2.2×10⁻⁶) and iPSC-Ns and miNs (p=8.66×10⁻¹⁴) (FIG. 2C). We also identified 630 genes which demonstrate similar and significant differences in alternative splicing between adult brain versus fetal neurons, also manifested in miNs versus iPSC-Ns. Some known age-associated splicing events captured between our aged and fetal samples include SCN2A, DCTN4, MAPRE3, and NEO1.

To specifically look at the 3R to 4R tau mRNA ratio, semi-quantitative PCR (sq-PCR) was used in which PCR is performed in a cDNA library with primers in exons 9 and 11, flanking exon 10, creating a long (4R) and short (3R) PCR product corresponding to the inclusion and exclusion of exon 10, respectively. consistent expression of 4R tau mRNA was found in miNs comparable to adult human brain samples, whereas all three types of fetal samples primarily expressed 3R tau (FIG. 2D). MiNs were found to express 50.3% of tau mRNA as 4R tau (FIG. 2D, right). Next, it was asked at what time point during the neuronal reprogramming tau expression begins, and in multiple fibroblast samples consistent, robust expression of both 3R and 4R tau transcripts were found starting at PID15 (FIG. 2E). Of note, this two-week point corresponds to the time that miNs start activating the neuronal program required for neuronal identity.

In addition to exon 10, MAPT exons 2 and 3 are also spliced under developmental control, with fetal samples only expressing a singular 0N isoform, whereas adult human brain expresses around 40% 0N, 50% 1N, and 10% 2N isoforms. Leafcutter determined that the inclusion of exon 2 was also differentially spliced between adult versus fetal samples, also reflected in miNs versus iPSC-Ns, where the fetal-aged samples showed no inclusion, but adult-aged samples expressed ˜50% 1N isoforms (FIG. 2F). To validate, sqPCR primers which flank exons 2 and 3 were designed, generating long, medium, and short PCR products depending on mRNA expression of 2N, 1N, and 0N isoforms, respectively. Fetal-aged samples only expressed 0N isoforms, whereas adult brain and miNs had equivalent expression of all three N isoforms (FIG. 2G).

To determine if the miRNAs were controlling the inclusion of exon 10, either non-specific miRNAs or miR-9/9*-124 were overexpressed in neural progenitor cells (NPCs) then differentiated them to iPSC-Ns through small molecule differentiation (Mahali and Karch, 2021). MiR-9/9*-124 had no effect on exon 10 inclusion and all iPSC-N samples expressed only 3R tau (Figure S2B). This data supports that miNs maintain the age of the starting cells (Huh et al., 2016), and this age drives exon 10 inclusion, and miNs recapitulate the endogenous 4R expression, generating the 1:1 3R:4R ratio seen in the adult brain.

What controls exon 10 inclusion has not been identified in the adult human brain context. It is possible that age-associated, differentially-expressed RNA binding proteins and splicing factors direct the inclusion of exon 10 in adult-aged samples. Bulk RNAseq between adult brain versus fetal neurons and miNs versus iPSC-Ns were compared pairwise and identified 88 differentially expressed genes identified by Ingenuity Pathway Analysis as splicing regulators.

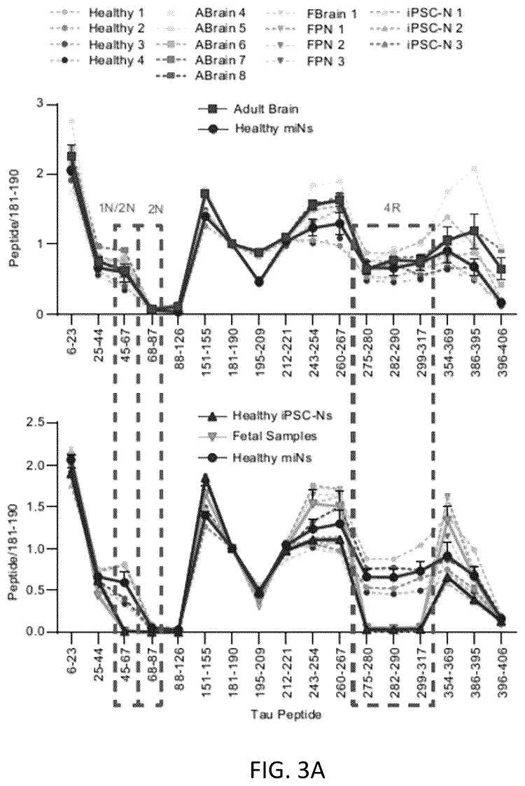

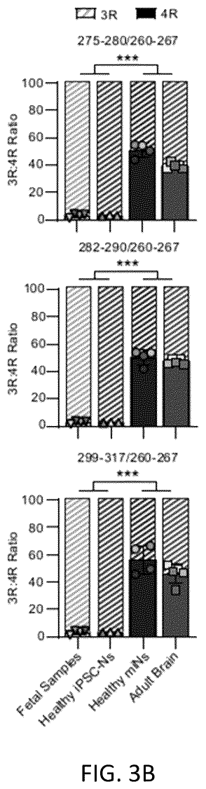

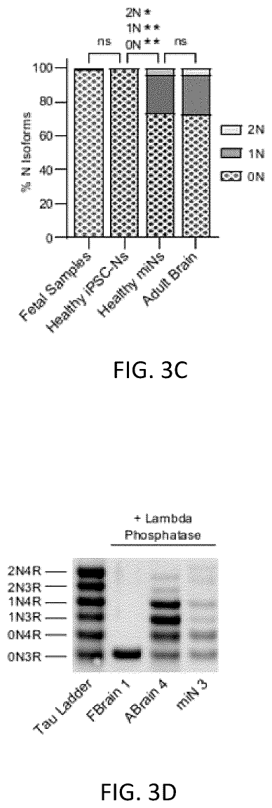

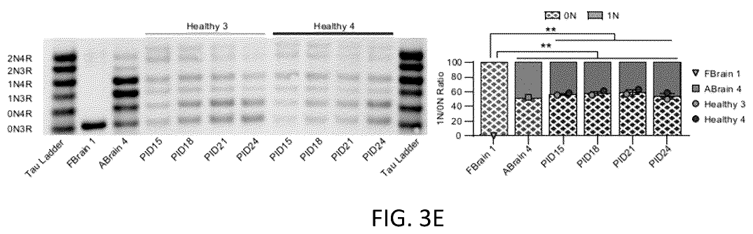

MiNs produce endogenous 4R tau protein similar to adult brain: To validate tau isoform expression at the protein level, mass spectrometry that can quantitatively and sensitively measure the tau isoforms in both cell culture and brain was performed on miNs from four independent adult individuals, four independent samples of fetal primary neurons or brain, adult brains from five individuals, and three iPSC-N replicates (FIG. 3A, solid line=mean). We quantified tau peptides across full length intracellular tau protein, including peptides specific for 4R (residues 275-280, 282-290, and 299-317). In iPSC-Ns and fetal samples, the 1N, 2N and 4R peptides were expressed at less than 3% of the sum of all tau isoforms (i.e. 0N+1N+2N, and 3R+4R, respectively) (FIG. 3A-FIG. 3D), consistent with previous literature showing iPSC-Ns recapitulate only fetal neuronal tau isoforms. Importantly, the 1N, 2N, and 4R peptide profiles of miNs showed significant enrichment over that of fetal samples, mirroring the tau profile of the adult brain samples (FIG. 3A-FIG. 3D). To specifically assay 3R:4R in miNs, the ratio of all three 4R-specific peptides were analyzed to the adjacent, constitutive R1 domain peptide (260-267). Remarkably, miNs display an approximately 1:1 3R to 4R ratio equivalent to that of the adult human brain, which drastically contrasted iPSC-Ns and fetal neurons (FIG. 3A-FIG. 3B). The presence of multiple tau isoforms in miNs and adult brain was confirmed via western blot, which differed with fetal brain expressing only 0N3R isoform (FIG. 3D). Further, a time course of western blot for tau in miNs at days 15-24 into reprogramming showed clear expression of both 3R and 4R isoforms at 1:1 3R:4R ratio throughout the immunoblots (FIG. 3E).

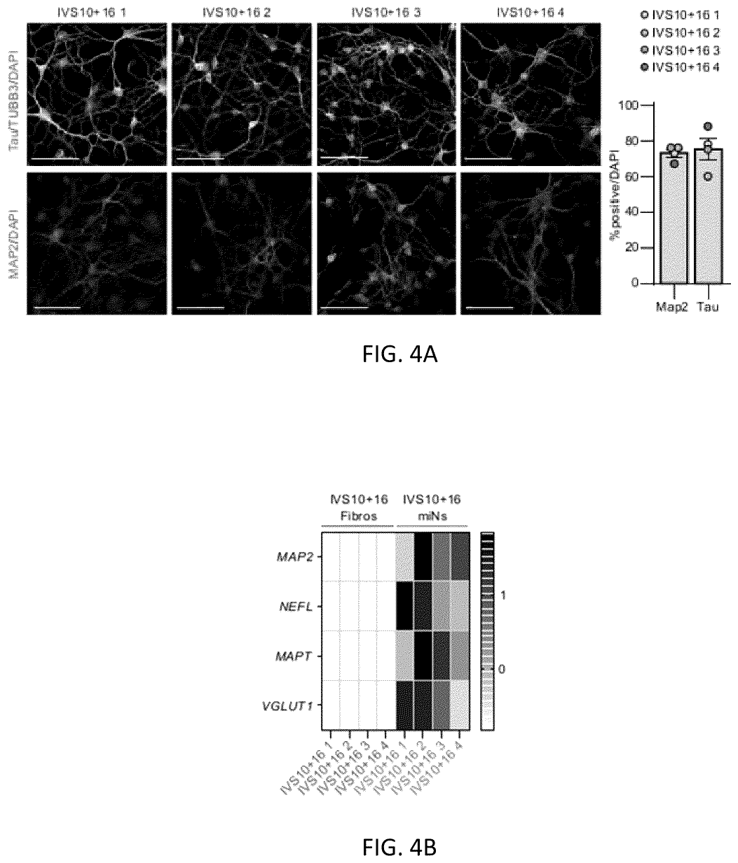

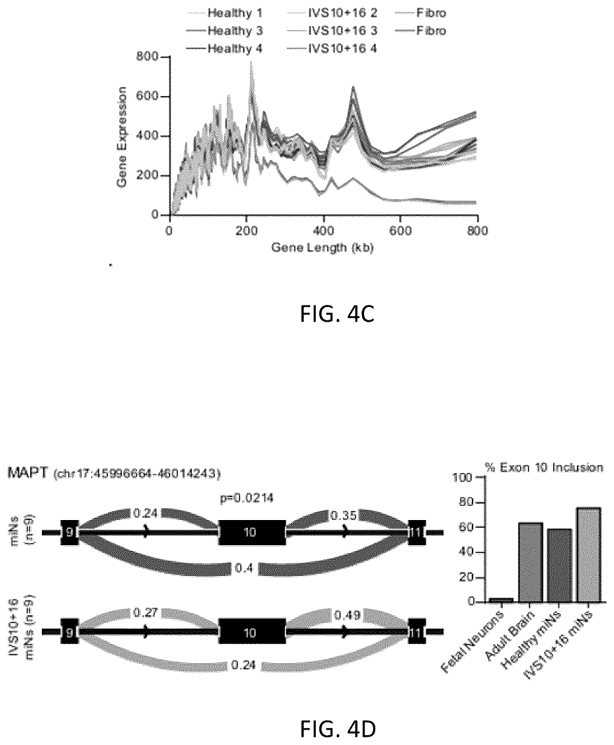

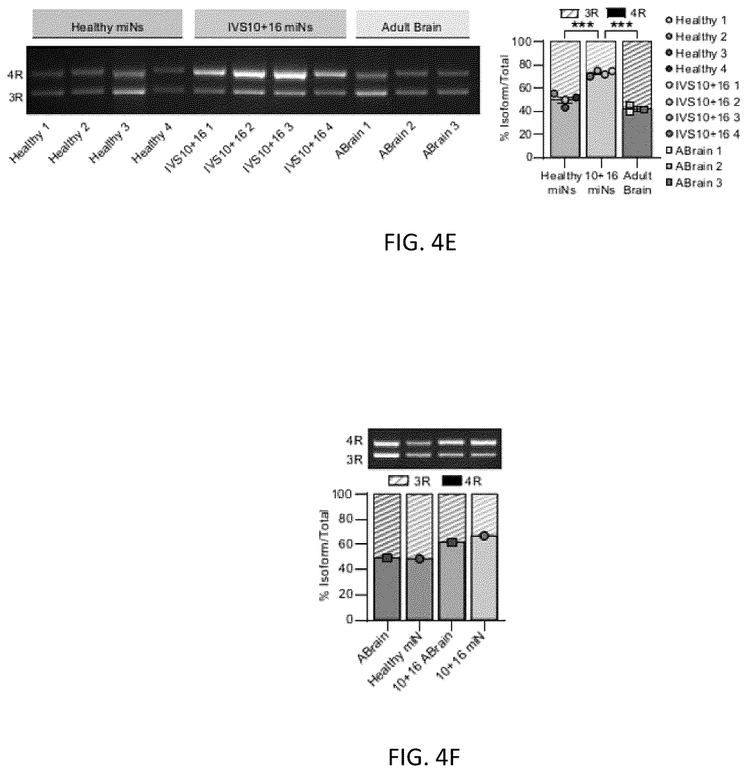

Directly reprogrammed miNs from tauopathy patient fibroblasts recapitulate abnormal 4R tau mRNA expression: Next, it was asked if the neuronal reprogramming would be sensitive to capture the increased 4R tau resulting from a point mutation at an intronic splice site known as IVS10+16 C>T (IVS10+16), which disrupts a stem-loop structure and biases splicing towards exon 10 inclusion. This familial mutation was shown to cause an approximately 2- to 4-fold increase in 4R tau mRNA and an imbalanced 3R:4R protein ratio, skewing towards increased 4R. First, fibroblast samples with confirmed mutation status from four symptomatic patients were directly converted, quantifying successful neuronal conversion, with 75% of DAPI-positive cells expressing MAP2 and tau (FIG. 4A). IVS10+16 miNs showed enrichment of neuronal genes over their starting fibroblasts (FIG. 4B). Neurons are different from most other cell types in their robust expression of long genes (>100 kb). Using LONGO, a gene length analysis R package, miNs from both healthy control and 6 IVS10+16 fibroblasts were successfully reprogrammed to neurons as seen by the forked trajectory between miNs and fibroblasts starting around 200 kb (FIG. 4C). Reprogrammed miNs had significantly higher long gene expression over the starting fibroblasts (P<0.001), but there was no significant difference in long gene expression between healthy and IVS10+16 miNs (p=0.177) indicating a similar conversion efficiency and no effect of the IVS10+16 mutation on neuronal reprogramming.

Leafcutter on bulk RNAseq to dissect the amount of exon 10 inclusion between healthy and IVS10+16 miNs was used, and found there was a significant increase in 4R tau in IVS10+16 samples (p=0.012) (FIG. 4D). This was validated using sq-PCR in which IVS10+16 miNs had a significant increase in exon 10 inclusion over that of healthy miNs and adult brain, with a 1.46-fold increase in their 4R:3R mRNA ratio (FIG. 4E). As this fold change differed from those published when this familial mutation was discovered, sqPCR on IVS10+16 human brain was performed and found the same fold change between IVS10+16 miNs and human brain over their corresponding healthy controls (FIG. 4F). As expected, N isoform expression ratios were not altered in IVS10+16 miNs.

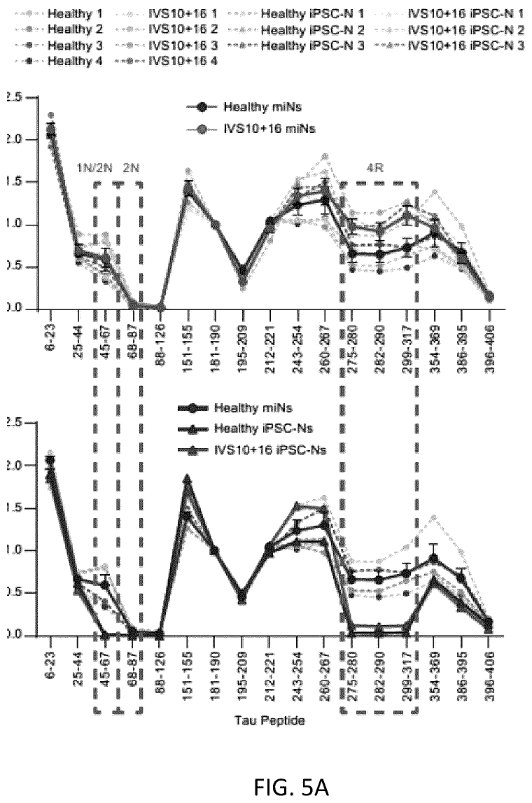

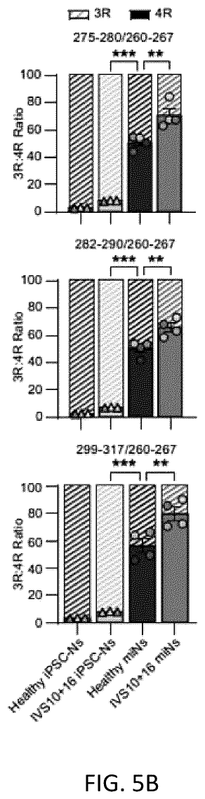

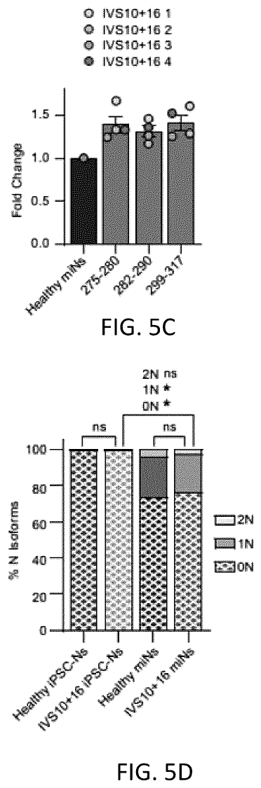

Increase in 4R tau protein in IVS10+16 miNs: To validate the increase in 4R mRNA on the protein level, mass spectrometry analyses on four independent patient-derived IVS10+16 miNs and isogenic IVS10+16 iPSC-Ns were performed, both of which demonstrated an increase in the 4R tau peptides over the age-matched, healthy counterparts (FIG. 5A, solid line=mean). IVS10+16 miNs had a significant increase in all three 4R-specific peptides over that of healthy miNs (FIG. 5B) IVS10+16 iPSC-Ns also showed significant increase in 4R tau over healthy iPSC-Ns but their final 4R tau expression is still far below that of the 3R tau level (FIG. 5A bottom: purple line, FIG. 5B: pink bars). The ratio of all three 4R-specific peptides matched the increase seen in mRNA, with a 1.31-1.41-fold increase in the IVS10+16 patient miNs over the healthy miNs (FIG. 5C). As expected, there was no significant difference in the profile of any other regions of tau including the ratio of 0N, 1N, or 2N isoforms between WT and IVS10+16 miNs (FIG. 5D).

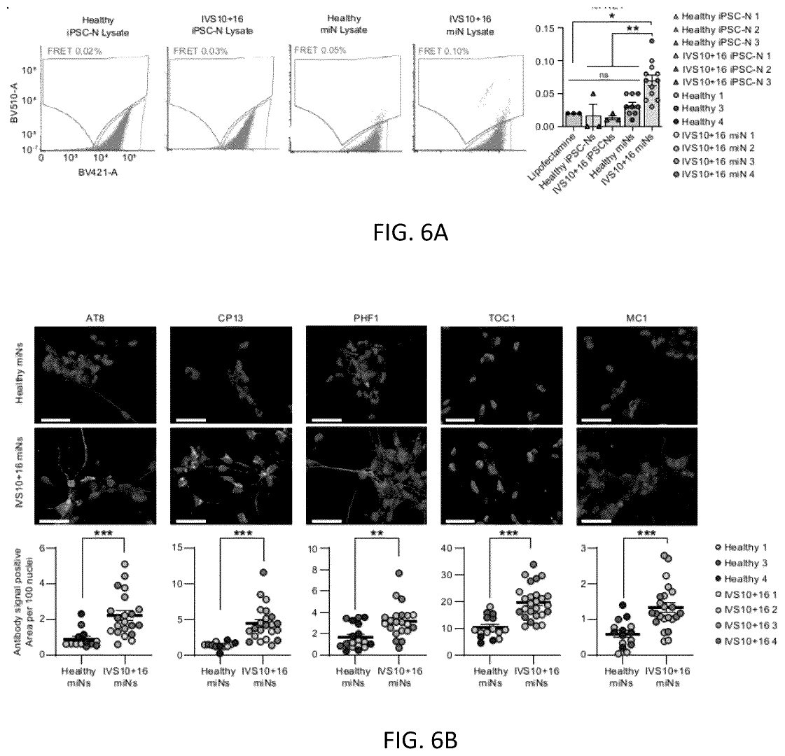

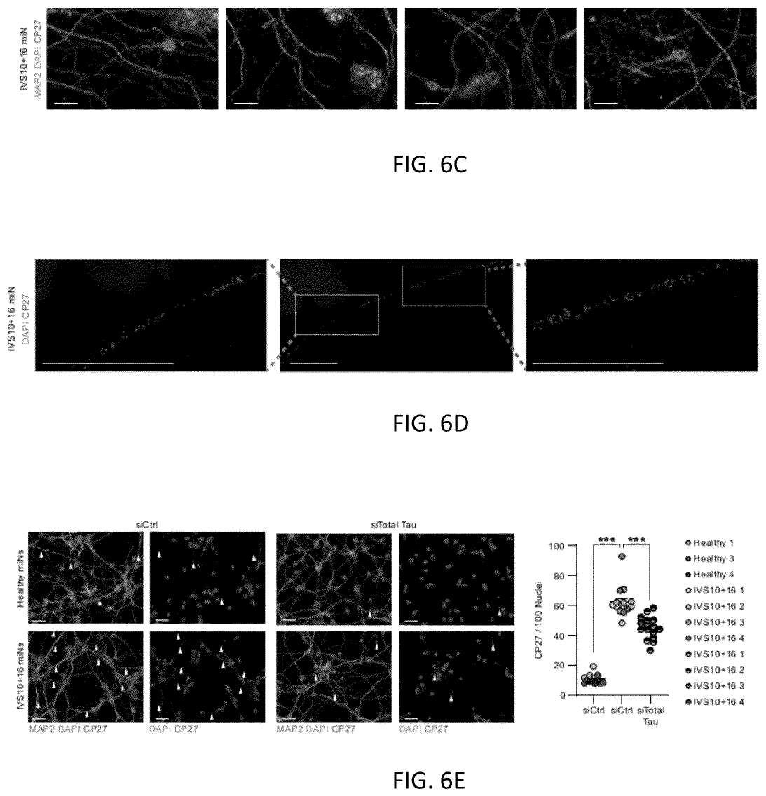

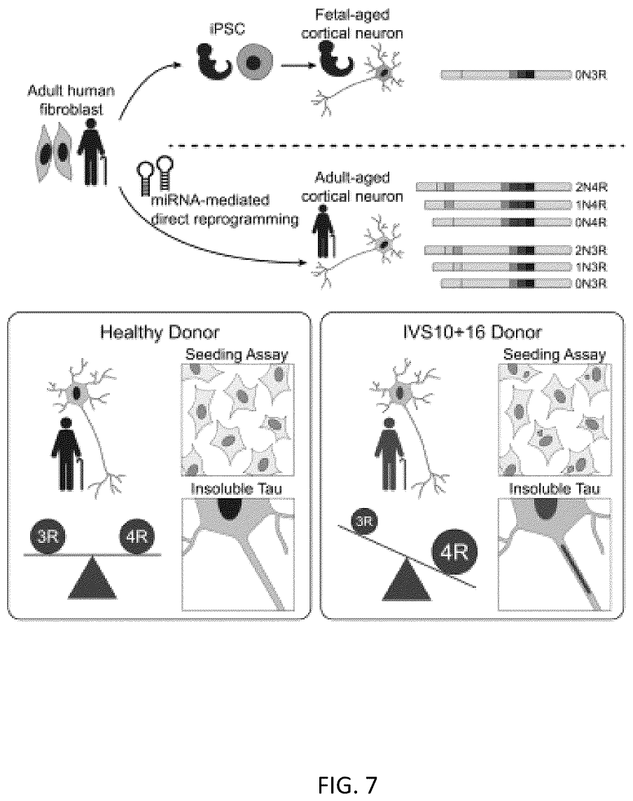

Increased insoluble tau and seeding capacity in IVS10+16 miNs: As a model system to endogenously express both the altered 3R:4R tau ratio seen in patient samples and have the advanced-age required for tau-associated phenotypes, IVS10+16 miNs were assessed for human tauopathy-specific pathologies. Previous studies have indicated that pathological tau has seeding capacity that precedes the formation of insoluble tau and which can be assayed using a tau-FRET Biosensor Assay. Patient-derived IVS10+16 miNs were found to produce seeding-competent tau, with a 50% increase in FRET-positive signal from IVS10+16 miN lysate than that of healthy miN lysate (FIG. 6A) indicating the presence of tau seeds. Importantly, control and isogenic IVS10+16 iPSC-Ns did not show any seeding capacity (FIG. 6A).

Next, methanol fixation method was used to remove soluble proteins and five tau-specific antibodies: AT8, (phospho-Ser202/Thr205), CP13 pS202 tau), PHF1 (phosphor-Ser396/Ser404), TOC1 (oligomeric tau), and MC1 (conformationally abnormal tau). All five antibodies showed a significant increase in signal in IVS10+16 miNs over that of healthy miNs, indicating the increased presence of insoluble tau in patient-derived miNs (FIG. 6B). No exogenous perturbation was applied to these cells; the insoluble tau developed through endogenous methods. This is in striking contrast to previously published data from IVS10+16 iPSC-Ns which were devoid of insoluble tau. We also observed thread-like tau staining in IVS10+16 miNs (FIG. 6C). Using stimulated emission depletion (STED) confocal microscope, higher resolution images of CP27-positive insoluble tau were obtained which demonstrated speckles of varying sizes along the neurites imaged (FIG. 6D). Insoluble tau signal was further confirmed to be specific and caused by the tau protein by applying tau siRNA to IVS10+16 miNs. Total tau was reduced by 30-40%, which led to significant reduction in the amount of insoluble tau (FIG. 6E).

### Discussion

The ability to study tau expression, isoform control, and age-associated disease pathologies will rely on a model system which endogenously recapitulates tau expression of the adult human brain. ReNcell-derived neurons display a dramatic increase in 4R tau transcript whose expression becomes enhanced in 3D culturing condition. Results shown here demonstrate miRNA-mediated neuronal conversion as a means to generate adult human neurons that mimic endogenous tau isoforms at both transcript and protein levels mirroring the 1-to-1 ratio of 3R:4R in the adult human brain. This ratio was strictly regulated in multiple independent donors. Using patient fibroblasts which harbor the IVS10+16 intronic mutation, it was found that miRNA-reprogramming were sensitive to capture the difference in mRNA levels caused by the genetic mutation and the subsequent increase in 4R protein levels that reflect fold increase at mRNA level.

The directly converted human neuron model with robust 4R tau expression raises interesting future questions, for instance, which age-associated splicing factors drive exon 10 inclusion. Engineered mouse models expressing the entire human MAPT genomic sequence deviated from the expected 1:1 ratio of 3R:4R, and showed a bias towards 3R, indicating there may exist splicing events and factors unique for 4R tau regulation in adult human neurons. Additionally, previous studies using tau minigenes to assay the regulation of tau exon 10 splicing in immortalized cell lines, has implicated over 100 putative splicing effectors including SRSF7, SFPQ, NOVA1, and DYRK1A, but these remain to be validated in an adult human neuronal context which could be addressed using miNs.

The maintenance of age is required for modeling adult-onset disorders supported by recently published recent study of age-associated phenotypes in patient-derived neurons. IVS10+16 patient-derived miNs develop increased insoluble tau inclusions and seed-competent tau, two defining characteristics of pathological tau from adult-onset tauopathy brains. The ability for a model to endogenously produce pathogenic tau will provide a system for assaying the prevention and clearing of insoluble tau. Of note, the model responded to the genetic knock-down of tau using siRNA which has therapeutic implications as anti-sense oligomer directed reduction of tau is in human clinical trials (Safety, Tolerability and Pharmacokinetics of Multiple Ascending Doses of NIO752 in Progressive Supranuclear Palsy).

Given the global increase in the aging population and the correlating increase in tauopathies, a model system that mirrors the adult human brain and its tau expression will provide an alternative avenue for studying these devastating diseases. The generation of human neurons that regulate tau splicing in the same way as the human adult brain and can replicate human tau-associated phenotypes represents a significant experimental advancement towards the investigation of tau-based pathology.

### Methods

Fibroblast cell lines: Healthy human fibroblasts were obtained from Coriell NINDS (AG04148—Male 56 yrs, AG08260—Male 61 yrs, AG08379—Female 60 yrs, and AG13369—Male 68 yrs) while IVS10+16 patient fibroblasts were acquired from Columbia University (ES046—Male 57 yrs) and University College London (UCL455—Male 53 yrs, UCL457—Male 52 yrs, and UCL497—Female 54 yrs). All fibroblasts were maintained in DMEM with 15% FBS for growth. Samples were grouped by MAPT genotype.

Adult human brain samples: Three biological replicate lots of adult human brain RNA (R1234066-50) (Lot C.210018—ABrain1 Male 29 yrs, Lot B.210080—ABrain2 Male 66 yrs, Lot B.811107—ABrain3 Female 78 yrs) was purchased from BioChain. Adult human brain protein samples include Novus purchased lysate (NB820—59177) (Lot C.111050—ABrain4 Male 82 yrs) and four previously published healthy samples (Lot 61732—ABrain5 Male 90 yrs, Lot 65241—ABrain6 Male 80 yrs, Lot 65318—ABrain7 Male 87 yrs, Lot 84868—ABrain8 Male 72 yrs) obtained from Washington University Alzheimer's Disease Research Center (ADRC). All participants were between A0B1 and A3B1 on the ABC Alzheimer's Disease lesion scoring system (none had clinical symptoms and they were not diagnosed with Alzheimer's Disease). IVS10+16 human brain (P2/08) from Queen Square House Brain Bank. Samples were grouped by MAPT genotype.

Fetal-aged human brain samples: Fetal human brain RNA (#1F01-50) (Lot 1333—Fbrain1 Male 21 weeks) was purchased from Cell Applications Inc. Fetal human neuron lysate (#1526) (Lot 2219—FNL1 sex and age not available) was purchased from ScienCell. Primary fetal neurons (#1520) (Lot 29207—FPN1 sex unknown 19 wks post-conception, Lot 28630—FPN2 Male 22 wks post conception, Lot 29390—FPN3 Male 18 wks post-conception) were purchased from ScienCell and were thawed and cultured in Neuronal Medium (ScienCell #1521). Samples were grouped by cellular age.

Purchased iPSC-derived neuron samples: For bulk and single cell RNA-sequencing and sqPCR, differentiated iPSC-derived Cortical Glutamatergic Neurons (Lot 200107—iPSC-CN1 Female) were purchased from BrainXell (#BX-0300) and cultured according to BrainXell protocol in DMEM with F12 and additional neuronal supplements. Samples were grouped by cellular age.

Lentiviral Production: Lentiviruses were generated as previously described (Church et al., 2021) with minor changes. To make supernatant virus, viral supernatant was collected, spun at 1200 g for 5 min at 4° C., then passed through a 0.45 μM filter. This supernatant was then aliquoted and directly frozen at −80° C. until used (less than 1 year after production date).

Direct Neuronal Reprogramming: Human fibroblasts were directly reprogrammed to miNs as previously described with modifications. Briefly, fibroblasts were transduced with supernatant lentivirus mix comprised of dox-inducible miR-9/9*-124, rtTA, and the transcription factor MYT1L. From PID1 to PID14 cells were treated with DAPT (2 uM) to increase neurite outgrowth and neuronal differentiation, and on PID 3, 6, 10, and 14, cells were also treated with the NEUROD1-activator ISX9 (10 uM) to push cortical fate. Fibroblasts and reprogramming cells were cultured in DMEM+10% FBS through replating at PID5. On PID6, cells were switched to Neurobasal-A with B27+(1000×) and Glutamax (500×), containing 1 μg/mL doxycycline, 200 μM dibutyl cyclic AMP, 1 mM valproic acid, 2 uM DAPT, 200 nM Ascorbic Acid, 10 ng/mL BDNF, 10 ng/mL NT-3, 1 μM retinoic acid, 10 uM ISX9, 100×RVC, and 3 μg/mL puromycin. Cells were half fed every 4 days and doxed every 4 days on an offsetting 2 day scheduled. On PID14, miNs were half fed using BrainPhys containing N2A and SM1 (StemCell) with the following goodies: 1 μg/mL doxycycline, 200 μM dibutyl cyclic AMP, 1 mM valproic acid, 2 uM DAPT, 200 nM Ascorbic Acid, 10 ng/mL BDNF, 10 ng/mL NT-3, and 1 μM retinoic acid.

iPSC Generation and Genome Engineering: Dermal fibroblasts from MAPT IVS10+16 carriers (GIH36) were transduced with non-integrating Sendai virus carrying OCT3/4, SOX2, KLF4, and cMYC (Life Technologies) as previously described (Karch et al., 2019). iPSC that were heterozygous for MAPT IVS10+16 were edited to WT (GIH36.2Δ1D01) using CRISPR/Cas9 as previously reported (Karch et al., 2019). Mutation status was confirmed by Sanger sequencing. Cell lines were maintained in mTesR medium (StemCell Technologies) on Matrigel. Cell lines were confirmed to be free of mycoplasma.

iPSC Differentiation: MAPT IVS10+16 iPSC (n=1) and isogenic controls (n=1) were differentiated into neural progenitor cells (NPCs) as previously described. Briefly, iPSC were dissociated with Accutase (Life Technologies). iPSCs were then plated at 65,000 cells per well in Neural Induction Media (NIM; Stem Cell Technologies) in a 96-well v-bottom plate to form neural aggregates. After 5 days, neural aggregates were plated on Poly-L-Ornithine (PLO) and laminin-coated plates to form neural rosettes. After 5 to 7 days, neural rosettes were isolated by enzymatic selection and cultured as NPCs. NPCs were cultured on PLO and laminin-coated plates and terminal differentiation was initiated with the addition of cortical maturation medium (Neurobasal-A (Life Technologies) supplemented with 1×B27 (Gibco), 20 ng/mL BDNF (Peprotech), 20 ng/mL GDNF (Peprotech), 0.5 mM cAMP (Sigma) and 1% L-glutamate (Sigma)). Neural cultures were maintained for six weeks.

Immunocytochemistry: Cells are fixed with either 4% PFA for 20 min and washed 3×PBS after fixation. Fixed cells were permeabilized for 10 min at RT in permeabilization buffer, blocked for 1 hr RT in 5% BSA/1% NGS, then stained in primary antibody in blocking buffer overnight 4° C. The next day, cells were washed 3× in PBS and placed in secondary antibody 1:1000 in blocking buffer for 1 hr room temp. Cells were washed 3× in PBS and stained with DAPI for 10 min RT, washed once with PBS, then mounted in ProLong Gold antifade Mountant. Primary antibodies used for the immunofluorescence imaging: mouse anti-NCAM (Santa Cruz, SC-106 1:50), rab anti-Tau (Aligent/DAKO, A002401-2 1:200), rab anti-TUBB3 (Biolegend, 802001 1:2000), mouse anti-TUBB3 (Biolegend, 801202 1:2000), chicken anti-TUBB3 (Novus, NB100-1612 1:1000), mouse anti-tau (CP27) (generously provided by Dr. Peter Davies, 1:400), mouse anti-pTau (PHF1) (generously provided by Dr. Peter Davies, 1:1000), mouse anti-pTau (AT8) (Invitrogen, MN1020, 1:1000), mouse anti-pTau (CP13) (generously provided by Dr. Peter Davies, 1:500), mouse anti-Tau (MC1) (generously provided by Dr. Peter Davies, 1:500), mouse anti-Tau (TOC1) (generously provided by Dr. Nicholas Kanaan, 1:500), rabbit anti-MAP2 (Cell Signaling, #4542 1:200), rabbit anti-MAP2 (Millipore, AB5622 1:1000), The secondary antibodies were goat anti-mouse, -rabbit, or -chicken IgG conjugated with Alexa-488, Alexa-594, or Alexa-647 (Invitrogen).

Immunostained images were taken using a Leica SP5× white light laser confocal system with Leica Application Suite (LAS) Advanced Fluorescence 2.7.3.9723. All antibodies were validated for functionality through negative control screening of fibroblasts. Composite images were stitched during acquisition in LAS software. Quantification of cell fate immunocytochemistry was performed using imageJ multi tool counter. Intact nuclei were used to count total cells, and MapAP2 and tau positive cells were defined by the presence of two or more fluorescent-positive neurites whose length is twice the size of the soma. N represents the total number of live cells counted per cell line, as outlined in figure legends. Percentages were calculated in Excel.

Quantitative PCR (qPCR): Total RNA was extracted from miNs using TRIzol (Invitrogen, USA) following manufacturer protocol. Reverse-transcription was performed with 150-200 ng of RNA with SuperScript IV First Strand Synthesis SuperMix (Invitrogen, USA). qPCR assay was run with SYBR Green PCR Master Mix and plate was run and analyzed on StepOnePlus Real-Time PCR System (AB Applied Biosystems, Germany). Each sample was run in triplicate and the mean of each sample is represented as a single data point. Expression values were calculated in Excel using the delta Ct method and Z-score calculation.

Single-cell RNAseq sample preparation: Reprogrammed cells, fibroblasts, and cultured iPSC-Ns were collected as previously published (Cates et al., 2020). Briefly, cells were washed once with 1×DPBS, 200 μL of 0.25% trypsin was added to the wells and plates were placed at 37° C. for 5 minutes. Wells were flooded with 500 μL warm 10% DMEM. Cells were collected in a 5 mL Eppendorf and centrifuged at 300 g for 5 minutes at 37° C. Supernatant was removed and the pellet was resuspended in 0.04% BSA in PBS and spun again. Pellet was resuspended in 0.04% BSA in PBS, cells were counted on a hemocytometer, and volume was adjusted to achieve 1,000 cells/μL. All samples were placed on ice and immediately brought to the Genome Technology Access Center at Washington University in Saint Louis (gtac.wustl.edu/).

10×: Single-cell RNAseq was performed on the 10× Genomics platform, using the Chromium Single Cell 3′ kits: Library & Gel Bead Kit v2 (PN-120237), Chip kit v2 (PN-120236), and 7 Multiplex Kit (PN-120262), following manufacturer-provided user guide. Agilent Bioanalyzer was used for cDNA library quantification.

Single-cell RNAseq data processing and analysis: The raw 10× reads were processed with the Cell Ranger count pipeline using default parameters (Cell Ranger v3.1.0, 10× Genomics. Reads were aligned to the hg38 reference index provided by 10× Genomics (refdata-cellranger-GRCh38 v3.0.0). Cell barcode and unique molecular identifier (UMI) were extracted and corrected from the feature library using the same methods as gene expression read processing. Feature-barcode matrices were generated by counting distinct UMIs of each gene within a given individual cell.

The R package, Seurat (v3.2.3) was used for quality control, analysis, and exploration of scRNA-seq data. We first removed cells where features less than 200 were detected and mitochondrial counts were high, then filtered features detected in cells less than 10. Seurat was used to remove unwanted variation from the gene expression by regressing out proportion of mitochondrial UMIs and overall UMIs. Highly variable genes were identified and used as input for dimensionality reduction via Principal Component Analysis (PCA). The resulting PCs and the correlated genes were examined to determine the number of components to be included in downstream analysis. These principal components were then used as inputs to cluster individual cells, using a K-nearest neighbor graph and the Louvain algorithm. The resulting cell clusters were visualized and explored using UMAP as a non-linear dimensional reduction technique.

Genotyping: Genomic DNA was extracted from fibroblasts using QIAamp DNA Mini Kit (Qiagen). PCR was performed using Phusion DNA polymerase (NEB) following the published protocol, with amplification primers at the annealing temperature of 63° C. for 35 cycles. PCR product was run on 1% agarose gel to confirm size and product was extracted using QIAquick Gel Extraction Kit (Qiagen). For restriction digest test, 500 ng of PCR product was then digested with NspI (NEB) then run on a 1% gel and imaged. For sequencing, purified PCR product was submitted to GeneWiz with sequencing primer and resulting tracks were visualized compared to control MAPT sequence.

RNAseq cell collection, sequencing, data processing, and analysis: MiNs were collected on PID21 in triplicate for RNA-sequencing using RNeasy Plus Micro Kit (Qiagen) according to the manufacturer's instructions. Cortical glutamatergic iPSC-Ns were purchased from BrainXell (BX-0300e), human adult whole brain RNA was purchased from Biochain (R1234035-50), and human fetal brain RNA sample was purchased from Cell Applications (1F01-50). Adult human brain and fetal brain RNA samples were run by GTAC through the NovaSeq6000 using SMARTer 150PE with 30 million reads per sample input. MiNs and iPSC-Ns were sequenced by DNALink (www.dnalink.com) on NovaSeq6000 using SMARTer 100PE and 40-50 million reads per sample input.

FastQC (bioinformatics.babraham.ac.uk/projects/fastqc) was used to determine sequencing quality and identify adapter contamination and FastQ files were trimmed using cutadapt for adapter contamination (if needed). Trimmed sequences were mapped to hg38 using STAR. Raw gene counts were extracted using deepTools multiBamSummary option -outRawCounts. Differential gene expression analysis was performed using DESeq2 normalizing to sequencing depth. Genes defined as differentially expressed had an adjusted p-value <0.05. Genes were categorized as RNA binding or splicing regulators by Ingenuity Pathway Analysis (QIAGEN).

Leafcutter data processing and analysis: New BAM files were generated by STAR using the --outSAMstrandField intronMotif option were used for Leafcutter, according to the published and recommended protocol (davidaknowles.github.io/leafcutter/). Samples were grouped by both cell-type and age and comparisons were performed pairwise, as required for Leafcutter analysis. Differentially spliced clusters and introns were defined by FDR<0.05, were exported using LeafViz by Jack Humphry (github.com/jackhump/leafviz), and loci colors were edited using illustrator (Adobe). Splicing line weights were linearly-scaled so all clusters had similar line weights for easier viewing. Per cluster, cryptic splicing events with less than 5% inclusion were removed.

Semi-quantitative PCR (sq-PCR): Semi-quantitative PCR was performed to quantify the ratio of 3R:4R mRNA. To perform sqPCR, cDNA was generated from RNA (SuperScript IV Reverse Transcriptase, ThermoFisher 18090010) then amplified using primers flanking exon 10 (forward 5′-AAGTCGCCGTCTTCCGCCAAG-3′ (SEQ ID NO: 1); reverse 5′-GTCCAGGGACCCAATCTTCGA-3′ (SEQ ID NO: 2)). The PCR product was then run on a 2% agarose gel with 381 bp and 288 bp fragments indicating 4R and 3R, respectively. Ratios were then calculated as previously described, via imageJ box plots and measure plots, summed pixel intensity values were exported to Excel, where each isoform value was divided by the summed total to generate percentage of each isoform. N isoform primers for sqPCR: forward 5′-TACGGGTTGGGGGACAGGAAACAT-3′ (SEQ ID NO: 3); reverse 5′-GGGGTGTCTCCAATGCCTGCTTCT-3′ (SEQ ID NO: 4).

Lysate Preparation for mass spectrometry analysis: MiNs were collected on PID 30. iPSC-Ns were collected after six weeks. Both were collected in the same manner. Briefly, media was removed and cells were washed once with DPBS. Fresh DPBS was added to the wells and cells were scraped off. Resuspended cells were pelleted at 1000 g for 5 min at RT, supernatant was removed, and pellets were frozen at −80° C. until use. Three different lots of Human Primary Neurons were purchased from ScienCell and plated according to manufacturer's instructions. Cells were cultured in Neuronal Medium (ScienCell #1521) for one week and collected same as the miNs and iPSC-Ns.

Adult brain frozen tissues was sliced via a cryostat at −20° C., from which 300-400 mg was sonicated in 4° C. buffer containing 25 mM tris-hydrochloride (pH 7.4), 150 mM sodium chloride, 10 mM ethylenediaminetetraacetic acid, 10 mM ethylene glycol tetraacedic acid, phosphatase inhibitor cocktail, and protease inhibitor cocktail, with final brain suspension at 0.3 mg/μL buffer. Suspension was cleared using centrifugation for 20 minutes at 11,000 g at 4° C. and the resulting supernatant was defined as “brain homogenate”.

Immunoprecipitation and mass spectrometry of tau isoforms: Mass spectrometry analyses of tau proteins were performed as previously described with some modifications. Cells and brain homogenates were diluted with PBS and 2% human serum albumin (Sigma D4197) respectively, and lysed with final concentration of 0.5% NP-40 and 2.5 mM guanidine. Tau protein was immunoprecipitated with mouse anti-Tau (Tau1) (provided by Dr. Nicholas Kanaan, 1.125 μg/sample), and mouse anti-human tau (HJ8.5) (provided by Dr. David Holtzman, 2.25 μg/sample), digested with trypsin, oxidized, desalted and subjected to nano-Acquity LC and MS analyses using Orbitrap Eclipse Tribrid Mass Spectrometer (Thermo Scientific). Mass spectrometry data were extracted using Skyline software.

All samples were spiked with full length N-labeled 2N4R recombinant tau as an internal standard and ratios of isoforms were calculated by using 2N and 4R isoform-specific peptides compared to constitutive peptides (all peptide numbers are in reference to 2N4R isoform). For 2N, the 2N-specific tryptic peptide 68-87 was divided by the common peptide 151-155 to obtain percentage of 2N. 1N isoform percentage was calculated by dividing a shared 1N/2N peptide, 45-67, by the common peptide 151-155, to obtain the cumulative percentage of 1N+2N isoforms. The previously calculated 2N percentage was subtracted from this shared 1N/2N percentage to calculate the 1N percentage. 0N percentage was calculated by subtracting both 1N and 2N percentages from 100. Percent 4R was calculated three ways by dividing each of the R2 region, 4R-specific peptides, 275-280, 282-290, and 299-317, by the constitutive adjacent R1 peptide, 260-267. 3R was calculated by subtracting the 4R percentage from 100.

Immunoblot analysis: Media was aspirated from wells, cells were washed with DPBS, and lysed in the plate with RIPA (Sigma #R0278) supplemented with complete Mini Protease Inhibitor Cocktail (Roche #11836153001). Lysate was collected and centrifuged at 13,000 g for 10 minutes at 4° C. BCA protein assay determined protein content and samples were normalized with RIPA/protease inhibitor. Samples were treated with Lambda Phosphatase (NEB #P0753L) for 3 hours at 30° C. LDS NuPAGE sample buffer with 5% β-mercaptoethanol was added and samples were heated to 95° C. for 3 minutes then loaded onto a NuPAGE 8% 20-well Midi gel (Invitrogen WG1002), with Tau Peptide Ladder (rPeptide T-1007-2). After running, the gel was transferred onto either 0.45 μm PVDF membrane for 2 hr at 400 mA. Membrane was both blocked and treated with primary antibodies in TBS/0.1% Tween20 overnight at 4° C.: rabbit anti-Tau antibody (Aligent/DAKO, A002401-2 1:1000-10,000). The next day, membrane was washed 3 times with TBST, then treated with peroxidase-conjugated goat anti-rabbit secondary antibodies in 5% Milk/TBS/0.1% Tween 20 for up to 3 hrs at RT. Membrane was washed 3 times with TBS/0.1% Tween 20 and developed with the ECL system (Thermo Scientific, #34076) and imaged on a Sapphire Imager (Azure Biosystems).

Biosensor cell culture methods: MiNs were harvested on PID 26 in 50 mM Tris-HCl, pH 7.5 150 mM NaCl. The lysates were sonicated for 4.5 minutes at 50% amplitude (QSonica, Q800R3 Sonicator). BCA assay was used determine protein concentration of protein. HEK biosensor Tau RD-CFP/YFP cell line, kindly provided by Dr. Marc Diamond, were cultured in 10 cm dish in DMEM (+Pyruvate, +D-glucose/D-glutamine) (Thermofisher) with 10% FBS and 1% Pen-Strep. The day before the seeding experiment, cells were replated in a 96-well plate at a density of 40,000 cells/well in 130 μL of media and left adhere over-night. The following day, cells were seeded with miN lysates; the seeding mixes were made by combining 17 μg of total protein lysate per well to 3.75 μL of Opti-MEM (Gibco) and 1.25 μL Lipofectamine 2000 (Invitrogen) for a total volume of 20 μL per well. Liposome preparations were incubated at room temperature for 30 min before adding to cells, and each condition was done in triplicate. Cells were incubated with seeding mixes for 72 h before the analysis.

FRET Flow Cytometry: After 72 h from the seeding, cells were washed in PBS (Gibco), harvested with 0.25% trypsin and fixed in 4% paraformaldehyde for 10 min, then resuspended in flow cytometry buffer (1 mM EDTA in PBS). The BD LSRFortessa™ Flow Cytometer was used to perform FRET flow cytometry as previously described. Briefly, to measure CFP and FRET, cells were excited with the 405 nm laser, and to measure YFP cells were excited with a 488 laser. Fluorescence was captured with a 405/50 nm, 525/50 nm and 525/50 nm filters respectively. To quantify FRET, a gating strategy similar to that previously described was used: first the CFP bleed-through into the YFP and FRET channels was compensated, and cells were gated in order to exclude YFP-positive only cells emitting in the FRET signal. A bivariate plot of FRET vs. CFP was made to assess the number of FRET-positive cells. The percentage of FRET (i.e., the number of FRET-positive cells per total cell count) and the Integrated FRET density (i.e. product of percent positivity and median fluorescence intensity) were used for the analyses. Data analysis was performed using FCS Express 7 Research software (De Novo Software).

Immunocytochemistry, imaging and analysis: Methanol fixed cells in DPBS had the liquid tipped off, and the cells were blocked with 200 μL/well of Intercept Blocking Buffer (LI-COR) containing 0.1% Triton for one hour at room temperature. The primary antibodies (see STAR methods) were prepared in Blocking Buffer and incubated overnight at 4° C. with gentle agitation. On the following day, the primary antibody was tipped off, and the cells were washed three times with 200 μL/well of DPBS. The secondary antibodies Goat anti-mouse IgG2b 647 Alexa Flour-conjugated antibody (A-21242) and Goat anti-rabbit 488 Alexa Flour-conjugated antibody (A-11008), as well as Hoechst 33342 (Invitrogen) were prepared at 1:1000 in Blocking Buffer and left for 1 hour at room temperature. The plates were then washed three times with 200 μL/well of DPBS, before being left in DPBS at 4° C. until high-content imaging using the Opera Phenix (Perkin Elmer). Images were taken using a 20× water objective, with 75 fields of view per well, and a Z-stack of 6 slices. The images were analysed using Perkin Elmer software Harmony as previously described. Briefly, stacks of each image were maximum projected and filtered using the sliding parabola to smoothen the background and get rid of the fluorescent noise. Subsequently, machine learning was used to define the tau positive area used as region in which the software was trained to identify the tau positive threads (for TOC1, only cytoplasmic signal was included in the analysis). Total nuclei were also detected and analyzed. At the end of the analysis results were exported in an Excel file divided by object (total nuclei and tau count) and well. The final readout was “Tau count normalized to the total nuclei count”. Any well with fewer than 800 nuclei was not included in analysis. n=total number of nuclei per line. Data was plotted in GraphPad.

STED Microscopy: STED microscopy was performed on a Leica STELLARIS 8 STED. Cells were methanol fixed as previously described. Primary antibodies were used at 1:1000. Secondary antibody was purchased from Abberior and used at 1:250.

siRNA treatment of miNs: MiNs were replated, as per the above protocol, at PID 5 on 24 well Sensoplate (Greiner, 662892) previously coated with poly-ornithine, laminin, and fibronectin. At PID 18, cells were treated with 1 uM of Dharmacon™ Accell™ siRNAs: Accell Human MAPT (4137) siRNA-SMARTpool (E-012488-00-0050) and Accell Non-targeting Pool (D-001910-10-05). Treatment was left on until PID 26 when cells were fixed with 100% methanol to extract soluble proteins, as described previously. In brief, the growth media was completely aspirated, and cells were washed twice with 200 μL/well of DPBS. Ice-cold 100% methanol was added 200 μL/well for 15 minutes at room temperature. Subsequently, the cells were washed three times with 200 μL/well of DPBS, with the final wash being left on for storage. Plates were kept at 4° C. until immunocytochemistry (ICC) was performed.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, a “population” of cells refers to a group of at least 2 cells, e.g. 2 cells, 3 cells, 4 cells, 10 cells, 100 cells, 1000 cells, 10,000 cells, 100,000 cells or any value in between, or more cells. Optionally, a population of cells can be cells which have a common origin, e.g. they can be descended from the same parental cell, they can be clonal, they can be isolated from or descended from cells isolated from the same tissue, or they can be isolated from or descended from cells isolated from the same tissue sample. Preferably, the population of hematopoietic progenitor cells is substantially purified. As used herein, the term “substantially purified” means a population of cells substantially homogeneous for a particular marker or combination of markers. By substantially homogeneous is meant at least 80%, at least 85%, at least 90%, at least 95%, at least 96%, at least 97%, at least 98%, at least 99% or more homogeneous for a particular marker or combination of markers.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The terms “composition” and “formulation” are used interchangeably.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs); and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is a non-human animal. In certain embodiments, the animal is a fish or reptile.

The term “administer,” “administering,” or “administration” refers to implanting, absorbing, ingesting, injecting, inhaling, or otherwise introducing a compound or cell described herein or generated as described herein, or a composition thereof, in or on a subject.

The terms “treatment,” “treat,” and “treating” refer to reversing, alleviating, delaying the onset of, or inhibiting the progress of a disease. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen and/or in light of detecting that the subject has a genotype associated with the disease). Treatment may also be continued after symptoms have resolved, for example, to delay or prevent recurrence.

The terms “condition,” “disease,” and “disorder” are used interchangeably.

The term “stem cell” refers to a vertebrate cell that has the ability both to self-renew, and to generate differentiated progeny. The ability to generate differentiated progeny may be described as pluripotent (see Morrison et al. (1997) Cell 88:287-298). “Embryonic stem cells” (ES cells) are pluripotent stem cells derived from the inner cell mass of a blastocyst, an early-stage preimplantation embryo. Pluripotency distinguishes embryonic stem cells from adult stem cells found in adults; while embryonic stem cells can generate all cell types in the body, adult stem cells are multipotent and can produce only a limited number of cell types.

The terms “induced neuronal cell,” “iN cell” “induced neuron,” or “iN” encompass cells of the neuronal lineage i.e. mitotic neuronal progenitor cells and post-mitotic neuronal precursor cells and mature neurons, that arise from a non-neuronal cell by experimental manipulation. Induced neuronal cells express markers specific for cells of the neuronal lineage, e.g. Tau, Tuj1, MAP2, NeuN, and the like, and may have characteristics of functional neurons, that is, they may be able to be depolarized, i.e. propagate an action potential, and they may be able to make and maintain synapses with other neurons.

The term “somatic cell” encompasses any cell in an organism that cannot give rise to all types of cells in an organism, i.e. it is not pluripotent. In other words, somatic cells are cells that have differentiated sufficiently that they will not naturally generate cells of all three germ layers of the body, i.e. ectoderm, mesoderm and endoderm.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). A “stem cell” is a cell characterized by the ability of self-renewal through mitotic cell division and the potential to differentiate into a tissue or an organ. Among mammalian stem cells, embryonic and somatic stem cells may be distinguished. Pluripotent stem cells, which include embryonic stem cells, embryonic germ cells and induced pluripotent cells, can contribute to tissues of a prenatal, postnatal or adult organism.

The terms “primary cells”, “primary cell lines”, and “primary cultures” are used interchangeably herein to refer to cells and cell cultures that have been derived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The terms “efficiency of reprogramming”, “reprogramming efficiency”, “efficiency of conversion”, or “conversion efficiency” are used interchangeably herein to refer to the ability of a culture of cells of one cell lineage to give rise to an induced cell of another cell lineage when contacted with a microRNA mediated neuronal cell induction agent of the invention. By “enhanced efficiency of reprogramming” or “enhanced efficiency of conversion” it is meant an enhanced ability of a culture of somatic cells to give rise to the induced neuronal cell when contacted with the reprogramming system relative to a culture of somatic cells that is not contacted with the reprogramming system, for example, an enhanced ability of a culture of cells to give rise to iN cells when contacted with a microRNA mediated neuronal cell induction agent relative to a culture of cells that is not contacted with the same agent. By enhanced, it is meant that the primary cells or primary cell cultures have an ability to give rise to the induced neuronal cells (e.g., iN cells) that is greater than the ability of a population that is not contacted with the induction agent, e.g., 150%, 200%, 300%, 400%, 600%, 800%, 1000%, or 2000% of the ability of the uncontacted population. In other words, the primary cells or primary cell cultures produce 1.5-fold or more, 2-fold or more, 3-fold or more, 4-fold or more, 6-fold or more, 8-fold or more, 10-fold or more, 20-fold or more, 30-fold or more, 50-fold or more, 100-fold or more, 200-fold or more the number of induced cells (e.g. iN cells) as the uncontacted population.

“Induced pluripotent stem cells”, abbreviated as PS cells, are a type of pluripotent stem cell artificially derived from a non-pluripotent cell, typically an adult somatic cell, by inducing expression of certain genes {e.g., injection of an expression construct). Induced pluripotent stem cells are identical in many respects to natural pluripotent stem cells, such as embryonic stem (ES) cells {e.g., in their physical properties). They may be the same in their expressions of certain stem cell genes and proteins, chromatin methylation patterns, doubling time, embryoid body formation, teratoma formation, viable chimera formation, and potency and differentiability. The term “induced pluripotent stem cell” encompasses pluripotent cells, that, like embryonic stem (ES) cells, can be cultured over a long period of time while maintaining the ability to differentiate into all types of cells in an organism. However, unlike ES cells (which are typically derived from the inner cell mass of blastocysts), PS cells are derived from differentiated somatic cells, that is, cells that have a narrower, more defined potential.

By “culturing” the cell means growing the cells in an artificial, in vitro environment. By “maintaining” means continuing to grow the cells in culture under suitable conditions until the pluripotency state of the cell is converted to a more naïve state.

“Cell line” refers to a population of largely or substantially identical cells, wherein the cells have often been derived from a single ancestor cell or from a defined and/or substantially identical population of ancestor cells. For example, a cell line may consist of descendants of a single cell. A cell line may have been or may be capable of being maintained in culture for an extended period (e.g., months, years, for an unlimited period of time). It will be appreciated that cells may acquire mutations and possibly epigenetic changes over time such that some individual cells of a cell line may differ with respect to each other. In some embodiments, at least 80%, 85%, 86%, 87%, 88%, 89%, 90%, 91%, 92%, 93%, 94%, 95%, 96%, 97%, 98%, or 99% of the cells of a cell line or cell culture are at least 95%, 96%, 97%, 98%, or 99% genetically identical. In some embodiments, at least 80%, 85%, 86%, 87%, 88%, 89%, 90%, 91%, 92%, 93%, 94%, 95%, 96%, 97%, 98%, or 99% of the cells of a cell line or cell culture express the same set of cell surface markers. The set of markers could be markers indicative of ground state (naïve) pluripotency or cell-type specific markers.

A “clone” refers to a cell derived from a single cell without change. It will be understood that if cells of a clone are subjected to different culture conditions or if some of the cells are subjected to genetic modification, the resulting cells may be considered distinct clones.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of modeling a neurodegenerative disease or a method of generating a neuron from an adult somatic cell comprising:
(i) providing an adult somatic cell, at least one miRNA capable of providing access to motor neuron genes in the adult somatic cell, and transcription factors;
(ii) providing the at least one miRNA to the adult somatic cell;
(iii) providing the transcription factors to the adult somatic cell, resulting in a transduced adult somatic cell; and
(iv) providing a NEUROD1-activator to the transduced adult somatic cell, resulting in the conversion of the adult somatic cell into a converted neuron.

2. The method of claim 1, wherein the transcription factors are selected from the group consisting of:
motor neuron transcription factors ISL LIM Homeobox 1 (ISL1) and/or LIM Homeobox 3 (LHX3); and striatal-enriched factors COUP-TF-Interacting Protein 2 (CTIP2), Distal-Less Homeobox 1 (DLX1), Distal-Less Homeobox 2 (DLX2), and/or Myelin Transcription Factor 1 Like (MYT1L (CDM)).

3. The method of claim 1, wherein the adult somatic cell is an adult human fibroblast of mesodermal origin.

4. The method of claim 1, wherein the miRNA is selected from miR-9/9* and miR-124 (miR-9/9*-124).

5. The method of claim 1, wherein the adult somatic cells are fibroblasts, the fibroblasts are transduced with supernatant lentivirus mix comprised of dox-inducible miR-9/9*-124 reverse tetracycline-controlled transactivator (rtTA), and the transcription factor MYT1L.

6. The method of claim 1, wherein from about day 1 to day 14 the somatic cells are treated with N—[N-(3,5-Difluorophenacetyl-L-alanyl)]-S-phenylglycine tbutyl ester (DAPT) to increase neurite outgrowth and neuronal differentiation.

7. The method of claim 6, wherein DAPT is in a concentration from about 0.2 μM to about 4 μM

8. The method of claim 1, wherein on about days 3, 6, 10, and 14, the cells are treated with a NEUROD1-activator to push cortical fate.

9. The method of claim 8, wherein the NEUROD1-activator is Isoxazole 9 (ISX9).

10. The method of claim 9, wherein ISX9 in in a concentration of about 1 μM to about 100 μM.

11. The method of claim 1, wherein the cells are cultured in a medium with Dulbecco's Modified Eagle Medium (DMEM) as the basal medium comprising Fetal Bovien Serum (FBS) through replating at day 5.

12. The method of claim 1, wherein on about day 6, the cells are contacted with a cell culture medium wherein the basal medium is Neurobasal-A with B27+, Glutamax, dibutyl cyclic AMP, valproic acid, DAPT, Ascorbic Acid, brain-derived neurotrophic factor (BDNF), Neurotrophin 3 (NT-3), retinoic acid, ISX9, RevitaCell Supplement (RVC), an antibiotic and optionally doxycycline when the expression of miR-9/9*-127 are under control of an inducible tet promoter.

13. The method of claim 12, wherein cells are half-fed every 4 days and doxed every 4 days on an offsetting 2 day scheduled.

14. The method of claim 1, wherein on about day 14, cells are half fed using a culture medium with BrainPhys as the basal medium with Neuro 2A (N2A), STEMCELL Modified-1 (SM1), dibutyl cyclic AMP, valproic acid, DAPT, Ascorbic Acid, BDNF, NT-3, retinoic acid and optionally doxycycline when the expression of miR-9/9*-127 are under control of an inducible tet promoter.

15. The method of claim 1, wherein the miRNA-induced neurons (miNs) recapitulate the expression of all six tau isoforms expressed in adult brains, with 4R tau establishing the 1:1 ratio with 3R tau.

16. The method of claim 1, wherein the miRNA-induced neurons (miNs) express endogenously 3R and 4R-tau levels analogous to an human adult brain.

17. The method of claim 1, wherein the miRNA-induced neurons (miNs) mirror the endogenous tau isoforms in health and disease (e.g., tauopathy).

18. The method of claim 1, wherein the miRNA-induced neurons (miNs) express the 4 repeat (4R) tau isoform.

19. The method of claim 1, wherein the miRNA-induced neurons (miNs) miNs express all six isoforms of tau having 3R/4R isoform ratio substantially equivalent to that detected in human adult brains.

20. The method of claim 1, wherein the miRNA-induced neurons (miNs) recapitulate tauopathy having increased 4R tau and the formation of insoluble tau with seeding activities.

21. The method of claim 1, wherein the converted neuron is a motor neuron or a medium spiny neuron (MSN).

22. The method of claim 1, wherein the miRNA or the transcription factors are expressed in the adult somatic cell comprising an adult somatic cell genome by viral vector transduction.

23. The method of claim 22, wherein a viral vector expresses miRNA and an anti-apoptotic gene, beneficial for neuronal conversion, under an inducible promoter.

24. The method of claim 22, wherein
the miRNA or the transcription factors are cloned into a lentiviral plasmid;
a lentivirus comprising a lentivirus genome is produced and the adult somatic cell is infected;
the lentivirus genome comprises the miRNA or the transcription factors and is transfected into the adult somatic cell genome, resulting in a transduced adult somatic cell; and
the miRNA or the transcription factors are stably expressed by the transduced adult somatic cell.

25. The method of claim 1, wherein the miRNA or the transcription factors are administered exogenously to the adult somatic cell.

26. The method of claim 1, wherein the miRNA
coordinates epigenetic and transcriptional changes resulting in neuronal cell fate conversion;
induces a generic neuronal state characterized by loss of fibroblast identity, presence of a pan-neuronal gene expression program, and absence of subtype specificity;
initiates subunit switching within BAF chromatin remodeling complexes while separately repressing neuronal cell-fate inhibitors REST, Co-REST, and SCP1; or
alters expression of genes involved in DNA methylation, histone modifications, chromatin remodeling, and chromatin compaction.

27. The method of claim 1, wherein the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron.

28. The method of claim 1, wherein
the converted neuron phenotypically resembles an endogenous motor neuron when compared using immunostaining analysis or gene expression profiling;
the converted neuron resembles the endogenous motor neuron when compared using electrophysiological tests or co-culture tests; or
the converted neuron retains donor age marks and positional information from the adult somatic cell.

29. The method of claim 1, wherein the neurodegenerative disease, disorder, or condition is selected from one or more of the group consisting of:
(i) a tauopathy;
(ii) a motor neuron disease;
(iii) spinal cord injury (SCI);
(iv) progressive supranuclear palsy (PSP); frontotemporal lobar degeneration (FTLD-TAU); corticobasal degeneration; or Alzheimer disease;
(v) Amyotrophic Lateral Sclerosis (ALS) or Spinal Muscular Atrophy (SMA); or
(vi) Huntington's Disease (HD) or Alzheimer's Disease (AD).

30. A method of screening a candidate drug for effectiveness in treating a tauopathy, neurodegenerative, or motor neuron disease comprising:
(i) providing a cellular platform, the cellular platform comprising neurons generated from fibroblasts of a subject with a neurodegenerative or motor neuron disease according to the method of any one of the preceding claims;
(ii) providing a candidate drug;
(iii) contacting the candidate drug and the cellular platform; and
(iv) assessing efficacy of the candidate drug.

31. The method of claim 30, wherein the cellular platform comprises cells obtained from a subject with a tauopathy, a motor neuron disease, Alzheimer's Disease (AD), Amyotrophic Lateral Sclerosis (ALS), Spinal Muscular Atrophy (SMA), Spinal Cord Injury (SCI), Huntington's Disease (HD), progressive supranuclear palsy (PSP), frontotemporal lobar degeneration (FTLD-TAU), or corticobasal degeneration.

32. The method of claim 30, wherein the efficacy is evaluated by monitoring the neurons for reversal of electrical impairment, spontaneous cell death, or stress-induced cell death.

33. The method of claim 32, wherein the subject has or is suspected of having a motor neuron disease, Alzheimer's Disease (AD), Amyotrophic Lateral Sclerosis (ALS), Spinal Muscular Atrophy (SMA), Spinal Cord Injury (SCI), or Huntington's Disease (HD).

34. The method of claim 32, wherein the subject has or is suspected of having a taupathy.

35. The method of claim 32, wherein the subject has or is suspected of having a taupathy selected from progressive supranuclear palsy (PSP); frontotemporal lobar degeneration (FTLD-TAU); corticobasal degeneration; or Alzheimer disease.

36. The method of claim 32, wherein the converted neuron is selected from the group consisting of: a motor neuron, a spinal motor neuron, a cortical neuron, a cortical-like neuron, a striatal neuron, a medium spiny neuron (MSN), a striatal medium spiny neuron (MSN), a dopaminergic neuron, a GABAergic neuron, a cholinergic neuron, serotonergic neuron, and a glutamatergic neuron.

37. A reprogrammed human neuron having a ratio (e.g., 1:1, non-1:1) of 4 repeat (4R) and 3 repeat (3R) isoforms consistent with healthy or pathological brain tissue generated according to any one of the preceding claims.

38. A method of generating a neuron cell from a non-neuronal somatic cell, the method comprising:
i) providing at least one non-neuronal somatic cell;
ii) expressing in the non-neuronal somatic cell a MYT1L transcription factor;
iii) culturing the cell from step ii) in the presence of a basal medium, serum and DAPT and adding ISX9 to the medium after about 3 days;
iv) culturing the cells from step iii) after about 5 days in the presence of a basal medium, B27, glutamax, dibutyl cyclic AMP, valproic acid, DAPT, Ascorbic Acid, BDNF, NT-3, retinoic acid, ISX9, RVC, and expressing in the cells miR-9/9*-127; and
v) culturing the cells from step iv) after about 9 days in the in the presence of a basal medium, N2A, SM1, dibutyl cyclic AMP, valproic acid, DAPT, Ascorbic Acid, BDNF, NT-3, and retinoic acid.

39. The method of claim 38, wherein the basal medium in step iii) is DMEM and the serum is FBS.

40. The method of claim 38, wherein the basal medium in step iv) is Neurobasal-A.

41. The method of claim 38, wherein the basal medium in step v) is BrainPhys.

42. A cell culture medium, wherein the cell culture medium is the medium of claim 39.

43. A cell culture medium, wherein the cell culture medium is the medium of claim 40.

44. A cell culture medium, wherein the cell culture medium is the medium of claim 41.

45. A kit for preparing the cell culture medium of claim 42, wherein the kit comprises individually packaged components, basal medium, and instructions for preparing the cell culture medium.

46. A kit for preparing the cell culture medium of claim 43, wherein the kit comprises individually packaged components, basal medium, and instructions for preparing the cell culture medium.

46. A kit for preparing the cell culture medium of claim 44, wherein the kit comprises individually packaged components, basal medium, and instructions for preparing the cell culture medium.

47. The method of claim 38, wherein the somatic cells are obtained from a healthy subject.

48. The method of claim 38, wherein the somatic cells are obtained from a subject diagnosed with a disease, disorder or at risk of a disease or disorder.

49. The method of claim 48, wherein the disease is a CNS disease.

50. The method of claim 49, wherein the disease is a tauopathy.

51. The method of any one of claims 38 or 47-50, wherein the somatic cell or generated neuron is genetically modified.

52. A population of neurons, which is produced by a method of any one of the preceding claims.

53. An in vitro cell culture system, comprising:
(i) a cell culture vessel; and
(ii) a population of neurons of claim 52.

54. A method for identifying an agent for treating a tauopathy or CNS disease, the method comprising:
(i) providing an in vitro cell culture system set forth in claim 53,
(ii) culturing the neuron cells in the presence of a candidate agent; and
(iii) identifying the candidate agent as an agent for treating a tauopathy or CNS disease, if the candidate agent maintains a cell phenotype or changes a cell phenotype to resemble a healthy control neuron.
